(19) United States
(12) Patent Application Publication
(10) Pub. No.: US 2007/0049626 A1
(43) Pub. Date: Mar. 1, 2007

(54) Treating premature ejaculation using gabapentin and pregabalin prodrugs

(76) Inventor: Pierre V. Tran, San Jose, CA (US)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/507,642

(22) Filed: Aug. 22, 2006

Related U.S. Application Data
(60) Provisional application No. 60/711,477, filed on Aug. 26, 2005.

(51) Int. Cl. A61K 31/325; A61K 31/40; A61K 31/44

(52) U.S. Cl. 514/357; 514/483; 514/408

(57) ABSTRACT

Disclosed herein are methods of using prodrugs of gabapentin and pregabalin, and pharmaceutical compositions thereof, to treat premature ejaculation in male humans, and pharmaceutical compositions of prodrugs of gabapentin and pregabalin useful in treating premature ejaculation.

This application claims the benefit of U.S. Provisional Application No. 60/711,477 filed Aug. 26, 2005, which is incorporated by reference herein in its entirety.

### 1. TECHNICAL FIELD

The methods and compositions disclosed herein relate generally to treating premature ejaculation in a male patient. More specifically, disclosed herein are methods of using prodrugs of gabapentin and pregabalin, and pharmaceutical compositions thereof, to treat premature ejaculation in male patients.

### 2. BACKGROUND

A normal erection occurs as a result of a coordinated vascular event in the penis, which is usually triggered neurally and includes vasodilation and smooth muscle relaxation in the penis and its supplying arterial vessels. Arterial inflow causes enlargement of the substance of the corpora cavernosa. Venous outflow is trapped by this enlargement, permitting sustained high blood pressures in the penis normally sufficient to cause rigidity. Muscles in the perineum also assist in creating and maintaining penile rigidity. Erection may also be induced centrally in the nervous system by sexual thoughts or fantasy, and is usually reinforced locally by reflex mechanisms. Erectile mechanics in women are substantially similar for the clitoris. In men, however, ejaculation typically occurs with an orgasm.

Premature ejaculation means persistent or recurrent ejaculation with minimal sexual stimulation before, upon, or shortly after penetration, and before the person wishes it. Premature ejaculation is one of the most common sexual complaints. It is estimated to affect up to 30 to 40 percent of men, i.e., approximately 36 million American men (Derogatis, L. R., Med. Aspects Hum. Sexuality, 14: 1168-76 (1980); Frank E., et al., Engl. J. Med., 299: 111-115 (1978); Schein, M., et al., Fam. Pract. Res. J., 7 (3): 122-134 (1988)). Ejaculation that occurs sooner than desired is often disappointing and can lead to other sexual dysfunctions including erectile difficulties, female inorgasmia, low sexual desire, and sexual aversion (Rust J., et al., Br. J. Psychiat., 152: 629-631 (1988)). Behavioral therapy, such as the Semans pause maneuver, the Masters and Johnson pause-squeeze technique or the Kaplan stop-start method, has been considered the gold standard for the treatment of premature ejaculation (Seftel, A. D., Altohob, S. E., “Premature Ejaculation”, Diagnosis and Management of Male Sexual Dysfunction, Edited by J. J. Mulcahy, New York, N.Y., Igaku-Shoin, (1997) Chapter 11, pages 196-203). While these techniques are harmless, usually painless, and are successful at rates of 60 to 95% (Seftel; Hawton, K., et al., Behav. Res. Ther., 24: 377 (1986)), they require partner cooperation and improvement is short-lived (Bancroft, J. and Coles, L., Brit. Med. J., 1: 1575 (1976) and De Amicus, L. A., et al., Arch. Sex. Behav., 14: 467 (1985)).

Premature ejaculation rarely has a physical cause, however, prostate gland inflammation or nervous system disorders may be involved. Recently, selective serotonin reuptake inhibitors (SSRIs), such as fluoxetine, paroxetine, sertraline and dapoxetine have been proposed for treating premature ejaculation (Merck Manual of Medical Information at 421-422, Home Edition, Merck Research Laboratories (1997)); see also U.S. Pat. No. 5,597,826 (sertraline), U.S. Pat. No. 5,276,042 (paroxetine), U.S. Pat. No. 5,672,612 (paroxetine) and U.S. Pat. No. 5,151,448 (fluoxetine); and Thor et al., WO 01/17521 (dapoxetine). Unfortunately, SSRIs are antidepressants and therefore their use to treat premature ejaculation has unwanted behavioral and/or mood altering side effects.

One γ-aminobutyric acid analog, gabapentin (1) which is sold under the tradename Neurontin®, has been used for the treatment of epileptic seizures and post-herpetic neuralgia. Another γ-aminobutyric acid analog, pregabalin (2) which is sold under the tradename Lyrica®, has been used for the treatment of peripheral neuropathic pain and epileptic seizures.

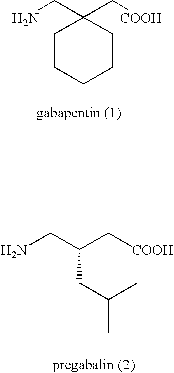

Gabapentin has also shown efficacy in controlled studies for treating neuropathic pain of varying etiologies and has been used to treat a number of other medical disorders (See Magnus, Epilepsia, 40: S66-72 (1999)). More recently, gabapentin and pregabalin have been disclosed to have utility in treating premature ejaculation (Taylor, Jr. et al., U.S. Patent Publication No. 2004/0176456).

One significant problem associated with the clinical use of gabapentin and pregabalin is their rapid systemic clearance. Consequently, these drugs require frequent dosing to maintain a therapeutic or prophylactic concentration in the systemic circulation (Bryans et al., Med. Res. Rev., 19: 149-177 (1999)). For example, dosing regimens of 300-600 mg doses of gabapentin administered three times per day are typically used for anticonvulsive therapy. Higher doses (1800-3600 mg/day in three or four divided doses) are typically used for the treatment of neuropathic pain states. The rapid systemic clearance and short half-life of gabapentin and pregabalin is problematic for the treatment of premature ejaculation; the drugs must be taken shortly before sexual activity occurs and/or must be retaken when sexual activity resumes, e.g., over the course of a day or evening.

Although oral sustained released formulations are conventionally used to reduce the dosing frequency of drugs that exhibit rapid systemic clearance, oral sustained release formulations of gabapentin and pregabalin have not been developed because these drugs are not absorbed via the large intestine. Rather, these compounds are typically absorbed in the small intestine by one or more amino acid transporters (e.g., the “large neutral amino acid transporter,” see Jezyk et al., Pharm. Res., 16: 519-526 (1999)). The limited residence time of both conventional and sustained release oral dosage forms in the proximal absorptive region of the gastrointestinal tract necessitates frequent daily dosing of conventional oral dosage forms of these drugs, and has prevented the successful application of sustained release technologies to gabapentin and pregabalin.

One method for overcoming rapid systemic clearance of gabapentin and pregabalin relies upon the use of gabapentin and pregabalin prodrugs that are absorbed from the large intestine/colon (Gallop et al., U.S. Pat. No. 6,818,787 and U.S. Patent Publication No. 2003/0083382, each of which is incorporated herein in its entirety). Such prodrugs may be administered using oral sustained release dosage forms and absorbed over wider regions of the gastrointestinal tract than the parent drug, and across the wall of the large intestine/colon where sustained release oral dosage forms typically spend a significant portion of gastrointestinal transit time. These prodrugs are typically converted to the parent gabapentin or pregabalin upon absorption in vivo. However, Gallop et al. only disclose using these prodrugs to treat epilepsy, depression, anxiety, psychosis, faintness attacks, hypokinesia, cranial disorders, neurodegenerative disorders, panic, pain (for example, neuropathic pain and muscular and skeletal pain), inflammatory disease (i.e., arthritis), insomnia, gastrointestinal disorders and ethanol withdrawal syndrome. Others have disclosed using certain of these prodrugs to treat hot flashes (Barrett et al., WO 2004/089289), urinary incontinence (Barrett, WO 2005/025562), and restless legs syndrome (Barrett et al., WO 2005/027850).

Current therapeutic agents for treating premature ejaculation either have significant behavior- and/or mood-altering side effects or are rapidly systemically cleared. Therefore, there is a need in the art for a new method of treating premature ejaculation without the side effect shortcomings of SSRI administration and rapid clearance/short half-life shortcomings of gabapentin and pregabalin administration.

### 3. DISCLOSURE

A method of treating premature ejaculation in a male patient in need of such treatment is disclosed herein. The method comprises administering to a male patient a therapeutically effective amount of a prodrug of at least one GABA analog chosen from compounds of Formula (I) and Formula (II): 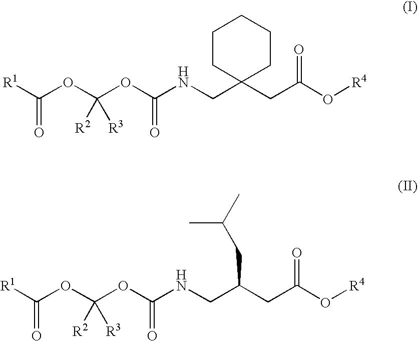
pharmaceutically acceptable salts thereof, pharmaceutically acceptable solvates of any of the foregoing, and pharmaceutically acceptable N-oxides of any of the foregoing, wherein:

R¹ is chosen from acyl, substituted acyl, alkyl, substituted alkyl, aryl, substituted aryl, arylalkyl, substituted arylalkyl, cycloalkyl, substituted cycloalkyl, cycloheteroalkyl, substituted cycloheteroalkyl, heteroalkyl, substituted heteroalkyl, heteroaryl, substituted heteroaryl, heteroarylalkyl, and substituted heteroarylalkyl;

R² and R³ are independently chosen from hydrogen, alkyl, substituted alkyl, alkoxycarbonyl, substituted alkoxycarbonyl, aryl, substituted aryl, arylalkyl, substituted arylalkyl, carbamoyl, substituted carbamoyl, cycloalkyl, substituted cycloalkyl, heteroalkyl, substituted heteroalkyl, cycloheteroalkyl, substituted cycloheteroalkyl, heteroaryl, substituted heteroaryl, heteroarylalkyl, and substituted heteroarylalkyl, or, R² and R³ together with the carbon atom to which they are bonded form a cycloalkyl, substituted cycloalkyl, cycloheteroalkyl or substituted cycloheteroalkyl ring; and

R⁴ is chosen from hydrogen, alkyl, substituted alkyl, aryl, substituted aryl, arylalkyl, substituted arylalkyl, cycloalkyl, substituted cycloalkyl, cycloheteroalkyl, substituted cycloheteroalkyl, heteroalkyl, substituted heteroalkyl, heteroaryl, substituted heteroaryl, heteroarylalkyl, and substituted heteroarylalkyl.

The prodrugs of Formulae (I) and (II) can administered in therapeutically effective amounts ranging from about 10 mg to about 5000 mg per day. In certain embodiments, the prodrugs are administered on an as needed basis ranging from about 0.25 to about 20 hours before anticipated sexual activity. The prodrugs can be administered orally, parenterally, subcutaneously, intravenously, intramuscularly, transdermally, intraperitoneally, intranasally, instillationally, intracavitarally, intravesical instillationally, intraocularly, intraarterially, intralesionally, or by application to mucous membranes.

### 4. DETAILED DESCRIPTION

### 4.1 Definitions

“Alkyl” by itself or as part of another substituent refers to a saturated or unsaturated, branched or straight-chain monovalent hydrocarbon radical derived by the removal of one hydrogen atom from a single carbon atom of a parent alkane, alkene or alkyne. Examples of alkyl groups include, but are not limited to, methyl; ethyls such as ethanyl, ethenyl, and ethynyl; propyls such as propan-1-yl, propan-2-yl, prop-1-en-1-yl, prop-1-en-2-yl, prop-2-en-1-yl(allyl), prop-1-yn-1-yl, prop-2-yn-1-yl, etc.; butyls such as butan-1-yl, butan-2-yl, 2-methyl-propan-1-yl, 2-methyl-propan-2-yl, but-1-en-1-yl, but-1-en-2-yl, 2-methyl-prop-1-en-1-yl, but-2-en-1-yl, but-2-en-2-yl, buta-1,3-dien-1-yl, buta-1,3-dien-2-yl, but-1-yn-1-yl, but-1-yn-3-yl, but-3-yn-1-yl, etc.; and the like.

The term “alkyl” is specifically intended to include groups having any degree or level of saturation, i.e., groups having exclusively single carbon-carbon bonds, groups having one or more double carbon-carbon bonds, groups having one or more triple carbon-carbon bonds and groups having mixtures of single, double and triple carbon-carbon bonds. Where a specific level of saturation is intended, the terms “alkanyl,” “alkenyl,” and “alkynyl” are used. In certain embodiments, an alkyl group comprises from 1 to 20 carbon atoms, in certain embodiments, from 1 to 6 carbon atoms, and in certain embodiments, from 1 to 3 carbon atoms.

“Alkanyl” by itself or as part of another substituent refers to a saturated branched or straight-chain alkyl radical derived by the removal of one hydrogen atom from a single carbon atom of a parent alkane. Examples of alkanyl groups include, but are not limited to, methanyl; ethanyl; propanyls such as propan-1-yl, and propan-2-yl (isopropyl), etc.; butanyls such as butan-1-yl, butan-2-yl(sec-butyl), 2-methyl-propan-1-yl(isobutyl), and 2-methyl-propan-2-yl(t-butyl), etc.; and the like.

“Alkenyl” by itself or as part of another substituent refers to an unsaturated branched or straight-chain alkyl radical having at least one carbon-carbon double bond derived by the removal of one hydrogen atom from a single carbon atom of a parent alkene. The group may be in either the cis or trans conformation about the double bond(s). Examples of alkenyl groups include, but are not limited to, ethenyl; propenyls such as prop-1-en-1-yl, prop-1-en-2-yl, prop-2-en-1-yl(allyl), and prop-2-en-2-yl, butenyls such as but-1-en-1-yl, but-1-en-2-yl, 2-methyl-prop-1-en-1-yl, but-2-en-1-yl, but-2-en-1-yl, but-2-en-2-yl, buta-1,3-dien-1-yl, and buta-1,3-dien-2-yl, etc.; and the like.

“Alkynyl” by itself or as part of another substituent refers to an unsaturated branched or straight-chain alkyl radical having at least one carbon-carbon triple bond derived by the removal of one hydrogen atom from a single carbon atom of a parent alkyne. Examples of alkynyl groups include, but are not limited to, ethynyl; propynyls such as prop-1-yn-1-yl, and prop-2-yn-1-yl, etc.; butynyls such as but-1-yn-1-yl, but-1-yn-3-yl, but-3-yn-1-yl, etc.; and the like.

“Acyl” by itself or as part of another substituent refers to a radical —C(O)R³⁰, where R³⁰ is chosen from hydrogen, alkyl, cycloalkyl, cycloheteroalkyl, aryl, arylalkyl, heteroalkyl, heteroaryl, and heteroarylalkyl as defined herein. Representative examples include, but are not limited to formyl, acetyl, cyclohexylcarbonyl, cyclohexylmethylcarbonyl, benzoyl, benzylcarbonyl, and the like.

“Alkoxy” by itself or as part of another substituent refers to a radical —OR³¹ where R³¹ is chosen from an alkyl and cycloalkyl group as defined herein. Representative examples include, but are not limited to, methoxy, ethoxy, propoxy, butoxy, cyclohexyloxy, and the like.

“Alkoxycarbonyl” by itself or as part of another substituent refers to a radical —C(O)OR³² where R³² is chosen from an alkyl and cycloalkyl group as defined herein. Representative examples include, but are not limited to, methoxycarbonyl, ethoxycarbonyl, propoxycarbonyl, butoxycarbonyl, cyclohexyloxycarbonyl, and the like.

“Aryl” by itself or as part of another substituent refers to a monovalent aromatic hydrocarbon radical derived by the removal of one hydrogen atom from a single carbon atom of a parent aromatic ring system. Typical aryl groups include, but are not limited to, groups derived from aceanthrylene, acenaphthylene, acephenanthrylene, anthracene, azulene, benzene, chrysene, coronene, fluoranthene, fluorene, hexacene, hexaphene, hexalene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, trinaphthalene, and the like. In certain embodiments, an aryl group comprises from 6 to 20 carbon atoms, for example, from 6 to 12 carbon atoms.

“Arylalkyl” by itself or as part of another substituent refers to an acyclic alkyl radical in which one of the hydrogen atoms bonded to a carbon atom, typically a terminal or sp³ carbon atom, is replaced with an aryl group. Typical arylalkyl groups include, but are not limited to, benzyl, 2-phenylethan-1-yl, 2-phenylethen-1-yl, naphthylmethyl, 2-naphthylethan-1-yl, 2-naphthylethen-1-yl, naphthobenzyl, 2-naphthophenylethan-1-yl and the like. Where specific alkyl moieties are intended, the nomenclature arylalkanyl, arylalkenyl and/or arylalkynyl is used. In certain embodiments, an arylalkyl group is (C₆-C₃₀) arylalkyl, e.g., the alkanyl, alkenyl or alkynyl moiety of the arylalkyl group is (C₁-C₁₀) and the aryl moiety is (C₆-C₂₀). In certain embodiments, an arylalkyl group is (C₆-C₂₀) arylalkyl, e.g., the alkanyl, alkenyl or alkynyl moiety of the arylalkyl group is (C₁-C₈) and the aryl moiety is (C₆-C₁₂).

“AUC” is the area under the plasma drug concentration-versus-time curve extrapolated from zero time to infinity.

“Carbamoyl” by itself or as part of another substituent refers to the radical —C(O)NR⁴⁰R⁴¹ where R⁴⁰ and R⁴¹ are independently chosen from hydrogen, alkyl, cycloalkyl, and aryl as defined herein.

“Cₘₐₓ” is the highest drug concentration observed in plasma following an extravascular dose of drug.

“Compounds” refers to prodrugs of GABA analogs, such as the gabapentin and pregabalin prodrugs of formulae (I) and (II), respectively, including any compounds encompassed by formulae (I) or (II). Compounds may be identified either by their chemical structure and/or chemical name. When the chemical structure and chemical name conflict, the chemical structure is determinative of the identity of the compound. The compounds described herein may comprise one or more chiral centers and/or double bonds and therefore, may exist as stereoisomers, such as double-bond isomers (i.e., geometric isomers), enantiomers, and diastereomers. Accordingly, the chemical structures depicted herein encompass all possible enantiomers and stereoisomers of the illustrated compounds including the stereoisomerically pure form (e.g., geometrically pure, enantiomerically pure or diastereomerically pure) and enantiomeric and stereoisomeric mixtures. Enantiomeric and stereoisomeric mixtures can be resolved into their component enantiomers or stereoisomers using separation techniques or chiral synthesis techniques well known to the skilled artisan. Compounds may also exist in several tautomeric forms including the enol form, the keto form and mixtures thereof. Accordingly, the chemical structures depicted herein encompass all possible tautomeric forms of the illustrated compounds. Compounds described herein also include isotopically labeled compounds where one or more atoms have an atomic mass different from the atomic mass conventionally found in nature. Examples of isotopes that may be incorporated into the compounds of the invention include, but are not limited to, ²H, ³H, ¹¹C, ¹³C, ¹⁴C, ¹⁵N, ¹⁸O, ¹⁷O, etc. Compounds may exist in unsolvated forms as well as solvated forms, including hydrated forms and as N-oxides. Certain compounds may exist in multiple crystalline or amorphous forms. All physical forms are equivalent for the uses contemplated herein. Further, it should be understood, when partial structures of the compounds are illustrated, that brackets indicate the point of attachment of the partial structure to the rest of the molecule.

“Cycloalkyl” by itself or as part of another substituent refers to a saturated or partially unsaturated cyclic alkyl radical. Where a specific level of saturation is intended, the nomenclature “cycloalkanyl” or “cycloalkenyl” is used. Typical cycloalkyl groups include, but are not limited to, groups derived from cyclopropane, cyclobutane, cyclopentane, cyclohexane, and the like. In certain embodiments, the cycloalkyl group is (C₃-C₁₀) cycloalkyl, for example, (C₃-C₇) cycloalkyl.

“Cycloheteroalkyl” by itself or as part of another substituent refers to a saturated or partially unsaturated cyclic alkyl radical in which one or more carbon atoms (and any associated hydrogen atoms) are independently replaced with the same or different heteroatom. Typical heteroatoms to replace the carbon atom(s) include, but are not limited to, N, P, O, S, Si, etc. Where a specific level of saturation is intended, the nomenclature “cycloheteroalkanyl” or “cycloheteroalkenyl” is used. Typical cycloheteroalkyl groups include, but are not limited to, groups derived from epoxides, azirines, thiiranes, imidazolidine, morpholine, piperazine, piperidine, pyrazolidine, pyrrolidine, quinuclidine, and the like.

“Heteroalkyl,” “heteroalkanyl,” “heteroalkenyl,” and “heteroalkynyl” by themselves or as part of another substituent refer to alkyl, alkanyl, alkenyl and alkynyl groups, respectively, in which one or more of the carbon atoms (and any associated hydrogen atoms) are independently replaced with the same or different heteroatomic groups. Typical heteroatomic groups include, but are not limited to, —O—, —S—, —O—O—, —S—S—, —O—S—, —NR⁴²R⁴³, -═N—N═-, —N═N—, —N═N—NR⁴⁴R⁴⁵, —PR⁴⁶—, —P(O)₂—, —POR⁴⁷, —O—P(O)₂—, —SO—, —SO₂—, —SnR⁴⁸R⁴⁹— and the like, where R⁴², R⁴³, R⁴⁴, R⁴⁵, R⁴⁶, R⁴⁷, R⁴⁸ and R⁴⁹ are independently chosen from hydrogen, alkyl, substituted alkyl, aryl, substituted aryl, arylalkyl, substituted arylalkyl, cycloalkyl, substituted cycloalkyl, cycloheteroalkyl, substituted cycloheteroalkyl, heteroalkyl, substituted heteroalkyl, heteroaryl, substituted heteroaryl, heteroarylalkyl, and substituted heteroarylalkyl.

“Heteroaryl” by itself or as part of another substituent refers to a monovalent heteroaromatic radical derived by the removal of one hydrogen atom from a single atom of a parent heteroaromatic ring system. Typical heteroaryl groups include, but are not limited to, groups derived from acridine, arsindole, carbazole, β-carboline, chromane, chromene, cinnoline, furan, imidazole, indazole, indole, indoline, indolizine, isobenzofuran, isochromene, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyran, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene, and the like. In certain embodiments, the heteroaryl group is a 5-20 membered heteroaryl, such as a 5-10 membered heteroaryl. In certain embodiments, heteroaryl groups are those derived from thiophene, pyrrole, benzothiophene, benzofuran, indole, pyridine, quinoline, imidazole, oxazole and pyrazine.

“Heteroarylalkyl” by itself or as part of another substituent refers to an acyclic alkyl radical in which one of the hydrogen atoms bonded to a carbon atom, typically a terminal or sp³ carbon atom, is replaced with a heteroaryl group. Where specific alkyl moieties are intended, the nomenclature heteroarylalkanyl, heteroarylalkenyl and/or heterorylalkynyl is used. In certain embodiments, the heteroarylalkyl group is a 6-30 membered heteroarylalkyl, e.g., the alkanyl, alkenyl, or alkynyl moiety of the heteroarylalkyl is 1-10 membered and the heteroaryl moiety is a 5-20-membered heteroaryl, such as a 6-20 membered heteroarylalkyl, e.g., the alkanyl, alkenyl or alkynyl moiety of the heteroarylalkyl is 1-8 membered and the heteroaryl moiety is a 5-12-membered heteroaryl.

“N-oxide” refers to the zwitterionic nitrogen oxide of a tertiary amine base.

“Parent aromatic ring system” refers to an unsaturated cyclic or polycyclic ring system having a conjugated π electron system. Specifically included within the definition of “parent aromatic ring system” are fused ring systems in which one or more of the rings are aromatic and one or more of the rings are saturated, partially unsaturated or unsaturated, such as, for example, fluorene, indane, indene, phenalene, etc. Examples of parent aromatic ring systems include, but are not limited to, aceanthrylene, acenaphthylene, acephenanthrylene, anthracene, azulene, benzene, chrysene, coronene, fluoranthene, fluorene, hexacene, hexaphene, hexalene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, trinaphthalene, and the like.

“Parent heteroaromatic ring system” refers to a parent aromatic ring system in which one or more carbon atoms (and any associated hydrogen atoms) are independently replaced with the same or different heteroatom. Typical heteroatoms that replace the carbon atoms include, but are not limited to, N, P, O, S, Si, etc. Specifically included within the definition of “parent heteroaromatic ring systems” are fused ring systems in which one or more of the rings are aromatic and one or more of the rings are saturated, partially unsaturated, or unsaturated, such as, for example, arsindole, benzodioxan, benzofuran, chromane, chromene, indole, indoline, xanthene, etc. Examples of parent heteroaromatic ring systems include, but are not limited to, arsindole, carbazole, β-carboline, chromane, chromene, cinnoline, furan, imidazole, indazole, indole, indoline, indolizine, isobenzofuran, isochromene, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyran, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene, and the like.

“Patient” refers to a male mammal, for example a human male. The terms “human” and “patient” are used interchangeably herein.

“Pharmaceutically acceptable” refers to approved or approvable by a regulatory agency of a federal or state government or listed in the U.S. Pharmacopoeia or other generally recognized pharmacopoeia for use in animals, for example, in humans.

“Pharmaceutically acceptable salt” refers to a salt of a compound, which possesses the desired pharmacological activity of the parent compound. Such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound is replaced by a metal ion, e.g., an alkali metal ion, an alkaline earth ion, or an aluminum ion; or coordinates with an organic base such as ethanolamine, diethanolamine, triethanolamine, N-methylglucamine, and the like.

“Pharmaceutically acceptable vehicle” refers to a diluent, adjuvant, excipient or carrier with which a compound of the invention is administered.

“Premature ejaculation” refers to a condition in a male patient characterized by persistent or recurrent ejaculation in a male patient with minimal sexual stimulation before, upon, or shortly after sexual penetration, and before the patient desires for it to occur.

“Prodrug” refers to either a derivative of gabapentin in accordance with Formula (I), or a derivative of pregabalin in accordance with Formula (II), that requires a transformation within the body to release the active gabapentin or pregabalin parent drug.

“Promoiety” refers to the organic moieties of Formulae (I) and (II) which are covalently bonded to the nitrogen atom of either gabapentin or pregabalin, converting the parent gabapentin or pregabalin drug into a prodrug. The promoieties are bonded to the attached to the parent drug via a bond that is cleaved by enzymatic and/or non-enzymatic means in vivo.

“Pharmaceutically acceptable solvate” refers to a molecular complex of a compound with one or more solvent molecules in a stoichiometric or non-stoichiometric amount. Such solvent molecules are those commonly used in the pharmaceutical art, which are known to be innocuous to recipient, e.g., water, ethanol, and the like. A molecular complex of a compound or moiety of a compound and a solvent can be stabilized by non-covalent intra-molecular forces such as, for example, electrostatic forces, van der Waals forces, or hydrogen bonds. The term “hydrate” refers to a complex where the one or more solvent molecules are water.

“Substituted” refers to a group in which one or more hydrogen atoms are independently replaced with the same or different substituent(s). Typical substituents include, but are not limited to, -M, —R⁶⁰, —O⁻, ═O, —OR⁶⁰, —SR⁶⁰, —S⁻, ═S, —NR⁶OR⁶, ═NR⁶⁰—CF₃, —CN, —OCN, —SCN, —NO, —NO₂, ═N₂, —N₃, —S(O)₂O, —S(O)₂OH, —S(O)₂R⁶⁰, —OS(O₂)O⁻, —OS(O)₂R⁶⁰, —P(O)(O⁻)₂, —P(O)(OR⁶⁰)(O⁻), —OP(O)(OR⁶⁰)(OR⁶¹), —C(O)R⁶⁰, —C(S)R⁶⁰, —C(O)OR⁶⁰, —C(O)NR⁶⁰R⁶¹, —C(O)O⁻, —C(S)OR⁶⁰, —NR⁶²C(O)NR⁶⁰R⁶¹, —NR⁶²C(S)NR⁶⁰R⁶¹, —NR⁶²C(NR⁶³)NR⁶⁰R⁶¹ and —C(NR⁶²)NR⁶⁰R⁶¹ where each M is independently a halogen; R⁶⁰, R⁶¹, R⁶² and R⁶³ are independently chosen from hydrogen, alkyl, substituted alkyl, alkoxy, substituted alkoxy, cycloalkyl, substituted cycloalkyl, cycloheteroalkyl, substituted cycloheteroalkyl, aryl, substituted aryl, heteroaryl or substituted heteroaryl, or optionally R⁶⁰ and R⁶¹ together with the nitrogen atom to which they are bonded form a cycloheteroalkyl or substituted cycloheteroalkyl ring; and R⁶² and R⁶³ are independently hydrogen, alkyl, substituted alkyl, aryl, cycloalkyl, substituted cycloalkyl, cycloheteroalkyl, substituted cycloheteroalkyl, aryl, substituted aryl, heteroaryl or substituted heteroaryl, or optionally R⁶² and R⁶³ together with the nitrogen atom to which they are bonded form a cycloheteroalkyl or substituted cycloheteroalkyl ring. In certain embodiments, substituents include -M, —R⁶⁰, ═O, —OR⁶⁰, —SR⁶⁰, —S⁻, ═S, —NR⁶⁰R⁶¹, ═NR⁶⁰, —CF₃, —CN, —OCN, —SCN, —NO, —NO₂, ═N₂, —N₃, —S(O)₂R⁶⁰, —OS(O₂)O⁻, —OS(O)₂R⁶⁰, —P(O)(O⁻)₂, —P(O)(OR⁶⁰)(O⁻), —OP(O)(OR⁶⁰)(OR⁶¹), —C(O)R⁶⁰, —C(S)R⁶⁰, —C(O)OR⁶⁰, —C(O)NR⁶OR⁶, —C(O)O⁻, —NR⁶²C(O)NR⁶⁰R⁶¹. In certain embodiments, substituents include -M, —R⁶⁰, ═O, —OR⁶⁰, —SR⁶⁰, —NR⁶⁰R⁶¹, —CF₃, —CN, —NO₂, —S(O)₂R⁶⁰, —P(O)(OR⁶⁰)(O⁻), —OP(O)(OR⁶⁰)(OR⁶¹), —C(O)R⁶⁰, —C(O)OR⁶⁰, —C(O)NR⁶OR⁶, —C(O)O⁻. In certain embodiments, substituents include -M, —R⁶⁰, ═O, —OR⁶⁰, —SR⁶⁰, —NR⁶⁰R⁶¹, —CF₃, —CN, —NO₂, —S(O)₂R⁶⁰, —OP(O)(OR⁶⁰)(OR⁶¹), —C(O)R⁶⁰, —C(O)OR⁶⁰, —C(O)O⁻, where R⁶⁰, R⁶¹ and R⁶² are as defined above.

In certain embodiments, each substituent group is independently chosen from halogen, —NH₂, —OH, —CN, —COOH, —C(O)NH₂, —C(O)OR⁵, and —NR⁵₃⁺, and each R⁵ is independently C₁₋₃ alkyl.

“Sustained release” refers to release of an agent from a dosage form at a rate effective to achieve a therapeutic or prophylactic amount of the agent, or active metabolite thereof, in the systemic blood circulation over a prolonged period of time relative to that achieved by oral administration of a conventional formulation of the agent. In some embodiments, release of the prodrug occurs over a sufficiently long period of time to achieve a therapeutic concentration of the gabapentin or pregabalin parent drug in the patient's blood plasma for a period of at least about 4 hours. In other embodiments, release of the prodrug occurs over a sufficiently long period of time to achieve a therapeutic concentration of the gabapentin or pregabalin parent drug in the patient's blood plasma for a period of at least about 8 hours. In still other embodiments, release of the prodrug occurs over a sufficiently long period of time to achieve a therapeutic concentration of the gabapentin or pregabalin parent drug in the patient's blood plasma for a period of at least about 12 hours.

“Tₘₐₓ” is the time to the maximum concentration (Cmax) of a drug in the plasma or blood of a patient following administration of a dose of the drug or prodrug thereof to the patient.

“Therapeutically effective amount” means the amount of a compound that, when administered to a male patient for treating premature ejaculation, is sufficient to effect such treatment. The “therapeutically effective amount” will vary depending on the compound, the disease and its severity and the age, weight, etc., of the male patient to be treated.

“Treating” or “treatment” of a disease refers to arresting or ameliorating a disease, disorder, or at least one of the clinical symptoms of a disease or disorder. In certain embodiments, “treating” or “treatment” refers to arresting or ameliorating at least one physical parameter of the disease or disorder, which may or may not be discernible by the patient. In certain embodiments, “treating” or “treatment” refers to inhibiting or controlling the disease or disorder, either physically (e.g., stabilization of a discernible symptom), physiologically (e.g., stabilization of a physical parameter), or both. In certain embodiments, “treating” or “treatment” refers to delaying, in some cases indefinitely, the onset of a disease or disorder.

Reference will now be made in detail to certain embodiments of the invention. While the invention will be described in conjunction with the disclosed embodiments, it will be understood that the disclosure is not intended to limit the invention to those disclosed embodiments. To the contrary, the spirit and scope of the invention is intended to cover alternatives, modifications, and equivalents as defined by the appended claims.

### 4.2 Gabapentin and Pregabalin Prodrugs

The disclosed method of treating premature ejaculation in a male patient comprises administering a therapeutically-effective amount of at least one gabapentin prodrug having the structure of Formula (I), a pregabalin prodrug having the structure of Formula (II): 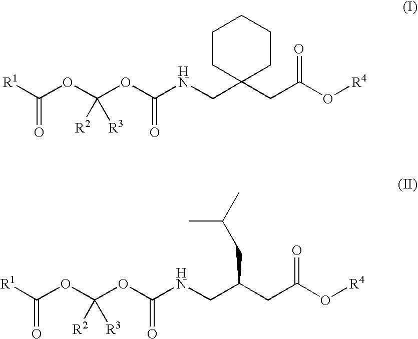
pharmaceutically acceptable salts thereof, pharmaceutically acceptable solvates of any of the foregoing, and pharmaceutically acceptable N-oxides of any of the foregoing, wherein:

R¹ is chosen from acyl, substituted acyl, alkyl, substituted alkyl, aryl, substituted aryl, arylalkyl, substituted arylalkyl, cycloalkyl, substituted cycloalkyl, cycloheteroalkyl, substituted cycloheteroalkyl, heteroalkyl, substituted heteroalkyl, heteroaryl, substituted heteroaryl, heteroarylalkyl, and substituted heteroarylalkyl;

R² and R³ are independently chosen from hydrogen, alkyl, substituted alkyl, alkoxycarbonyl, substituted alkoxycarbonyl, aryl, substituted aryl, arylalkyl, substituted arylalkyl, carbamoyl, substituted carbamoyl, cycloalkyl, substituted cycloalkyl, heteroaryl, substituted heteroaryl, heteroarylalkyl, and substituted heteroarylalkyl, or R² and R³ together with the carbon atom to which they are bonded form a cycloalkyl, substituted cycloalkyl, cycloheteroalkyl, or substituted cycloheteroalkyl ring; and

R⁴ is chosen from hydrogen, alkyl, substituted alkyl, aryl, substituted aryl, arylalkyl, substituted arylalkyl, cycloalkyl, substituted cycloalkyl, cycloheteroalkyl, substituted cycloheteroalkyl, heteroalkyl, substituted heteroalkyl, heteroaryl, substituted heteroaryl, heteroarylalkyl, and substituted heteroarylalkyl.

In certain embodiments, for example, when R⁴ is substituted alkyl, each substituent group is independently chosen from halogen, —NH₂, —OH, —CN, —COOH, —C(O)NH₂, —C(O)OR⁵, and —NR⁵₃⁺, and each R⁵ is independently C₁₋₃ alkyl.

In some embodiments, R² and R³ are independently hydrogen, alkyl, substituted alkyl, alkoxycarbonyl, aryl, arylalkyl, carbamoyl, cycloalkyl, substituted cycloalkyl, or heteroaryl. For example, R² is alkoxycarbonyl or carbamoyl and R³ is methyl. In other embodiments, R² and R³ are independently hydrogen, methyl, ethyl, propyl, isopropyl, butyl, isobutyl, sec-butyl, tert-butyl, cyclopentyl, cyclohexyl, methoxycarbonyl, ethoxycarbonyl, propoxycarbonyl, isopropoxycarbonyl, butoxycarbonyl, isobutoxycarbonyl, sec-butoxycarbonyl, tert-butoxycarbonyl, cyclohexyloxycarbonyl, phenyl, benzyl, phenethyl, or 3-pyridyl.

In still other embodiments, R² and R³ are independently hydrogen, alkanyl, substituted alkanyl, cycloalkanyl, or substituted cycloalkanyl. In still other embodiments, R² and R³ are independently hydrogen, alkanyl, or cycloalkanyl. In still other embodiments, R² and R³ are independently hydrogen, methyl, ethyl, propyl, isopropyl, butyl, isobutyl, sec-butyl, tert-butyl, cyclopentyl, or cyclohexyl. In still other embodiments, R² is methyl, ethyl, propyl, isopropyl, butyl, isobutyl, sec-butyl, tert-butyl, cyclopentyl, or cyclohexyl, and R³ is hydrogen, or R² is methyl and R³ is methyl.

In still other embodiments, R² and R³ are independently hydrogen, aryl, arylalkyl, or heteroaryl. In still other embodiments, R² and R³ are independently hydrogen, phenyl, benzyl, phenethyl, or 3-pyridyl. In still other embodiments, R² is phenyl, benzyl, phenethyl, or 3-pyridyl, and R³ is hydrogen.

In still other embodiments, R² and R³ are independently hydrogen, alkyl, substituted alkyl, alkoxycarbonyl, or carbamoyl. In still other embodiments, R² is alkoxycarbonyl or carbamoyl, and R³ is methyl. In still other embodiments, R² is methoxycarbonyl, ethoxycarbonyl, isopropoxycarbonyl, butoxycarbonyl, isobutoxycarbonyl, sec-butoxycarbonyl, tert-butoxycarbonyl, or cyclohexyloxycarbonyl, and R³ is methyl.

In still other embodiments, R² and R³ together with the carbon atom to which they are bonded form a cycloalkyl, substituted cycloalkyl, cycloheteroalkyl, or substituted cycloheteroalkyl ring. In still other embodiments, R² and R³ together with the carbon atom to which they are bonded form a cycloalkyl ring. In still other embodiments, R² and R³ together with the carbon atom to which they are bonded form a cyclobutyl, cyclopentyl, or cyclohexyl ring.

In still other embodiments of compounds of Formula (I), R¹ is acyl, substituted acyl, alkyl, substituted alkyl, aryl, arylalkyl, cycloalkyl, or heteroaryl. In still other embodiments, R¹ is methyl, ethyl, propyl, isopropyl, butyl, isobutyl, sec-butyl, pentyl, isopentyl, sec-pentyl, neopentyl, 1,1-dimethoxyethyl, 1,1-diethoxyethyl, 1-(1,3-dioxolan-2-yl)-ethyl, 1-(1,3-dioxan-2-yl)-ethyl, 1,1-dimethoxypropyl, 1,1-diethoxypropyl, 1-(1,3-dioxolan-2-yl)-propyl, 1-(1,3-dioxan-2-yl)-propyl, 1,1-dimethoxybutyl, 1,1-diethoxybutyl, 1-(1,3-dioxolan-2-yl)-butyl, 1-(1,3-dioxan-2-yl)-butyl, 1,1-dimethoxybenzyl, 1,1-diethoxybenzyl, 1-(1,3-dioxolan-2-yl)-benzyl, 1-(1,3-dioxan-2-yl)-benzyl, 1,1-dimethoxy-2-phenethyl, 1,1-diethoxy-2-phenethyl, 1-(1,3-dioxolan-2-yl)-2-phenethyl, 1-(1,3-dioxan-2-yl)-2-phenethyl, acetyl, propionyl, butyryl, benzoyl, phenacetyl, phenyl, 4-methoxyphenyl, benzyl, phenethyl, styryl, cyclopropyl, cyclobutyl, cyclopentyl, cyclohexyl, or 3-pyridyl.

In still other embodiments, R¹ is acyl or substituted acyl. In still other embodiments, R¹ is acetyl, propionyl, butyryl, benzoyl, or phenacetyl.

In still other embodiments, R¹ is alkanyl or substituted alkanyl. In still other embodiments, R¹ is methyl, ethyl, propyl, isopropyl, butyl, isobutyl, sec-butyl, pentyl, isopentyl, sec-pentyl, neopentyl, 1,1-dimethoxyethyl, 1,1-diethoxyethyl, 1-(1,3-dioxolan-2-yl)-ethyl, 1-(1,3-dioxan-2-yl)-ethyl, 1,1-dimethoxypropyl, 1,1-diethoxypropyl, 1-(1,3-dioxolan-2-yl)-propyl, 1-(1,3-dioxan-2-yl)-propyl, 1,1-dimethoxybutyl, 1,1-diethoxybutyl, 1-(1,3-dioxolan-2-yl)-butyl, 1-(1,3-dioxan-2-yl)-butyl, 1,1-dimethoxybenzyl, 1,1-diethoxybenzyl, 1-(1,3-dioxolan-2-yl)-benzyl, 1-(1,3-dioxan-2-yl)-benzyl, 1,1-dimethoxy-2-phenethyl, 1,1-diethoxy-2-phenethyl, 1-(1,3-dioxolan-2-yl)-2-phenethyl or 1-(1,3-dioxan-2-yl)-2-phenethyl. In still other embodiments, R¹ is methyl, ethyl, propyl, isopropyl, butyl, 1,1-dimethoxyethyl, or 1,1-diethoxyethyl.

In still other embodiments, R¹ is aryl, arylalkyl or heteroaryl. In still other embodiments, R¹ is phenyl, 4-methoxyphenyl, benzyl, phenethyl, styryl, or 3-pyridyl.

In still other embodiments, R¹ is cycloalkyl or substituted cycloalkyl. In still other embodiments, R¹ is cyclopropyl, cyclobutyl, cyclopentyl, or cyclohexyl.

In still other embodiments, R¹ is acyl, substituted acyl, alkyl, substituted alkyl, aryl, arylalkyl, cycloalkyl or heteroaryl and R² and R³ are independently hydrogen, alkyl, substituted alkyl, alkoxycarbonyl, aryl, arylalkyl, carbamoyl, cycloalkyl, or heteroaryl. In certain embodiments, R¹ is acyl, alkyl, substituted alkyl, aryl, arylalkyl, cycloalkyl or heteroaryl, R² is alkoxycarbonyl, or carbamoyl, and R³ is methyl. In still other embodiments, R¹ is methyl, ethyl, propyl, isopropyl, butyl, isobutyl, sec-butyl, pentyl, isopentyl, sec-pentyl, neopentyl, 1,1-dimethoxyethyl, 1,1-diethoxyethyl, 1-(1,3-dioxolan-2-yl)-ethyl, 1-(1,3-dioxan-2-yl)-ethyl, 1,1-dimethoxypropyl, 1,1-diethoxypropyl, 1-(1,3-dioxolan-2-yl)-propyl, 1-(1,3-dioxan-2-yl)-propyl, 1,1-dimethoxybutyl, 1,1-diethoxybutyl, 1-(1,3-dioxolan-2-yl)-butyl, 1-(1,3-dioxan-2-yl)-butyl, 1,1-dimethoxybenzyl, 1,1-diethoxybenzyl, 1-(1,3-dioxolan-2-yl)-benzyl, 1-(1,3-dioxan-2-yl)-benzyl, 1,1-dimethoxy-2-phenethyl, 1,1-diethoxy-2-phenethyl, 1-(1,3-dioxolan-2-yl)-2-phenethyl, 1-(1,3-dioxan-2-yl)-2-phenethyl, acetyl, propionyl, butyryl, benzoyl, phenacetyl, phenyl, 4-methoxyphenyl, benzyl, phenethyl, styryl, cyclopropyl, cyclobutyl, cyclopentyl, cyclohexyl, or 3-pyridyl and R² and R³ are independently hydrogen, methyl, ethyl, propyl, isopropyl, butyl, isobutyl, sec-butyl, tert-butyl, cyclopentyl, cyclohexyl, methoxycarbonyl, ethoxycarbonyl, propoxycarbonyl, isopropoxycarbonyl, butoxycarbonyl, isobutoxycarbonyl, sec-butoxycarbonyl, tert-butoxycarbonyl, cyclohexyloxycarbonyl, phenyl, benzyl, phenethyl, or 3-pyridyl. In still other embodiments, R¹ is methyl, ethyl, propyl, isopropyl, butyl, isobutyl, sec-butyl, 1,1-dimethoxyethyl, 1,1-diethoxyethyl, 1,1-dimethoxybenzyl, 1,1-diethoxybenzyl, 1,1-dimethoxy-2-phenethyl, 1,1-diethoxy-2-phenethyl, acetyl, propionyl, butyryl, benzoyl, phenacetyl, phenyl, 4-methoxyphenyl, benzyl, phenethyl, cyclohexyl, or 3-pyridyl, and R² and R³ are independently hydrogen, methyl, ethyl, propyl, isopropyl, butyl, isobutyl, sec-butyl, tert-butyl, cyclopentyl, cyclohexyl, methoxycarbonyl, ethoxycarbonyl, isopropoxycarbonyl, cyclohexyloxycarbonyl, phenyl, benzyl, phenethyl, or 3-pyridyl.

In still other embodiments, R¹ is acyl, substituted acyl, alkyl, substituted alkyl, aryl, substituted aryl, arylalkyl, substituted arylalkyl, cycloalkyl, substituted cycloalkyl, cycloheteroalkyl, substituted cycloheteroalkyl, heteroalkyl, substituted heteroalkyl, heteroaryl, substituted heteroaryl, heteroarylalkyl, or substituted heteroarylalkyl, and R² and R³ together with the atom to which they are bonded form a cycloalkyl, substituted cycloalkyl, cycloheteroalkyl, or a substituted cycloheteroalkyl ring. In still other embodiments, R¹ is acyl, substituted acyl, alkyl, substituted alkyl, aryl, arylalkyl, cycloalkyl, or heteroaryl, and R² and R³ together with the atom to which they are bonded form a cycloalkyl, or a substituted cycloalkyl ring. In still other embodiments, R¹ is methyl, ethyl, propyl, isopropyl, butyl, isobutyl, sec-butyl, pentyl, isopentyl, sec-pentyl, neopentyl, 1,1-dimethoxyethyl, 1,1-diethoxyethyl, 1-(1,3-dioxolan-2-yl)-ethyl, 1-(1,3-dioxan-2-yl)-ethyl, 1,1-dimethoxypropyl, 1,1-diethoxypropyl, 1-(1,3-dioxolan-2-yl)-propyl, 1-(1,3-dioxan-2-yl)-propyl, 1,1-dimethoxybutyl, 1,1-diethoxybutyl, 1-(1,3-dioxolan-2-yl)-butyl, 1-(1,3-dioxan-2-yl)-butyl, 1,1-dimethoxybenzyl, 1,1-diethoxybenzyl, 1-(1,3-dioxolan-2-yl)-benzyl, 1-(1,3-dioxan-2-yl)-benzyl, 1,1-dimethoxy-2-phenethyl, 1,1-diethoxy-2-phenethyl, 1-(1,3-dioxolan-2-yl)-2-phenethyl, 1-(1,3-dioxan-2-yl)-2-phenethyl, acetyl, propionyl, butyryl, benzoyl, phenacetyl, phenyl, 4-methoxyphenyl, benzyl, phenethyl, styryl, cyclopropyl, cyclobutyl, cyclopentyl, cyclohexyl, or 3-pyridyl, and R² and R³ together with the atom to which they are bonded form a cyclobutyl, cyclopentyl, or a cyclohexyl ring.

In still other embodiments, R¹ is acyl or substituted acyl, and R² and R³ are independently hydrogen, alkyl, substituted alkyl, alkoxycarbonyl, substituted alkoxycarbonyl, aryl, substituted aryl, arylalkyl, substituted arylalkyl, carbamoyl, substituted carbamoyl, cycloalkyl, substituted cycloalkyl, heteroaryl, or substituted heteroaryl. In certain embodiments, R¹ is acyl or substituted acyl, R² is alkoxycarbonyl, substituted alkoxycarbonyl, carbamoyl, or substituted carbamoyl, and R³ is methyl. In still other embodiments, R¹ is acetyl, propionyl, butyryl, benzoyl, or phenacetyl, and R² and R³ are independently hydrogen, alkyl, substituted alkyl, alkoxycarbonyl, substituted alkoxycarbonyl, aryl, substituted aryl, arylalkyl, substituted arylalkyl, carbamoyl, substituted carbamoyl, cycloalkyl, substituted cycloalkyl, heteroaryl, or substituted heteroaryl. In certain embodiments, R¹ is acetyl, propionyl, butyryl, benzoyl, or phenacetyl, R² is alkoxycarbonyl or carbamoyl, and R³ is methyl.

In still other embodiments, R¹ is alkanyl or substituted alkanyl, and R² and R³ are independently hydrogen, alkyl, substituted alkyl, alkoxycarbonyl, substituted alkoxycarbonyl, aryl, substituted aryl, arylalkyl, substituted arylalkyl, carbamoyl, substituted carbamoyl, cycloalkyl, substituted cycloalkyl, heteroaryl, or substituted heteroaryl. In certain embodiments, R¹ is alkanyl or substituted alkanyl, R² is alkoxycarbonyl, substituted alkoxycarbonyl, carbamoyl, or substituted carbamoyl, and R³ is methyl.

In still other embodiments, R¹ is methyl, ethyl, propyl, isopropyl, butyl, isobutyl, sec-butyl, pentyl, isopentyl, sec-pentyl, neopentyl, 1,1-dimethoxyethyl, 1,1-diethoxyethyl, 1-(1,3-dioxolan-2-yl)-ethyl, 1-(1,3-dioxan-2-yl)-ethyl, 1,1-dimethoxypropyl, 1,1-diethoxypropyl, 1-(1,3-dioxolan-2-yl)-propyl, 1-(1,3-dioxan-2-yl)-propyl, 1,1-dimethoxybutyl, 1,1-diethoxybutyl, 1-(1,3-dioxolan-2-yl)-butyl, 1-(1,3-dioxan-2-yl)-butyl, 1,1-dimethoxybenzyl, 1,1-diethoxybenzyl, 1-(1,3-dioxolan-2-yl)-benzyl, 1-(1,3-dioxan-2-yl)-benzyl, 1,1-dimethoxy-2-phenethyl, 1,1-diethoxy-2-phenethyl, 1-(1,3-dioxolan-2-yl)-2-phenethyl, or 1-(1,3-dioxan-2-yl)-2-phenethyl, and R² and R³ are independently hydrogen, alkyl, substituted alkyl, alkoxycarbonyl, substituted alkoxycarbonyl, aryl, substituted aryl, arylalkyl, substituted arylalkyl, carbamoyl, substituted carbamoyl, cycloalkyl, substituted cycloalkyl, heteroaryl, or substituted heteroaryl. In certain embodiments, R¹ is methyl, ethyl, propyl, isopropyl, butyl, isobutyl, sec-butyl, pentyl, isopentyl, sec-pentyl, neopentyl, 1,1-dimethoxyethyl, 1,1-diethoxyethyl, 1-(1,3-dioxolan-2-yl)-ethyl, 1-(1,3-dioxan-2-yl)-ethyl, 1,1-dimethoxypropyl, 1,1-diethoxypropyl, 1-(1,3-dioxolan-2-yl)-propyl, 1-(1,3-dioxan-2-yl)-propyl, 1,1-dimethoxybutyl, 1,1-diethoxybutyl, 1-(1,3-dioxolan-2-yl)-butyl, 1-(1,3-dioxan-2-yl)-butyl, 1,1-dimethoxybenzyl, 1,1-diethoxybenzyl, 1-(1,3-dioxolan-2-yl)-benzyl, 1-(1,3-dioxan-2-yl)-benzyl, 1,1-dimethoxy-2-phenethyl, 1,1-diethoxy-2-phenethyl, 1-(1,3-dioxolan-2-yl)-2-phenethyl, or 1-(1,3-dioxan-2-yl)-2-phenethyl, R² is alkoxycarbonyl or carbamoyl, and R³ is methyl.

In still other embodiments, R¹ is aryl, substituted aryl, arylalkyl, substituted arylalkyl, heteroaryl, or substituted heteroaryl, and R² and R³ are independently hydrogen, alkyl, substituted alkyl, alkoxycarbonyl, substituted alkoxycarbonyl, aryl, substituted aryl, arylalkyl, substituted arylalkyl, carbamoyl, cycloalkyl, substituted cycloalkyl, heteroaryl, or substituted heteroaryl. In certain embodiments, R¹ is aryl, substituted aryl, arylalkyl, substituted arylalkyl, heteroaryl, or substituted heteroaryl, R² is alkoxycarbonyl, substituted alkoxycarbonyl, carbamoyl, or substituted carbamoyl, and R³ is methyl. In still other embodiments, R¹ is phenyl, 4-methoxyphenyl, benzyl, phenethyl, styryl, or 3-pyridyl and R² and R³ are independently hydrogen, alkyl, substituted alkyl, alkoxycarbonyl, substituted alkoxycarbonyl, aryl, substituted aryl, arylalkyl, substituted arylalkyl, carbamoyl, cycloalkyl, substituted cycloalkyl, heteroaryl, or substituted heteroaryl. In certain embodiments, R¹ is phenyl, 4-methoxyphenyl, benzyl, phenethyl, styryl, or 3-pyridyl, R² is alkoxycarbonyl or carbamoyl, and R³ is methyl.

In still other embodiments, R¹ is cycloalkyl or substituted cycloalkyl, and R² and R³ are independently hydrogen, alkyl, substituted alkyl, alkoxycarbonyl, substituted alkoxycarbonyl, aryl, substituted aryl, arylalkyl, substituted arylalkyl, carbamoyl, cycloalkyl, substituted cycloalkyl, heteroaryl, or substituted heteroaryl. In certain embodiments, R¹ is cycloalkyl or substituted cycloalkyl, R² is alkoxycarbonyl, substituted alkoxycarbonyl, carbamoyl, or substituted carbamoyl, and R³ is methyl. In still other embodiments, R¹ is cyclopropyl, cyclobutyl, cyclopentyl, or cyclohexyl, and R² and R³ are independently hydrogen, alkyl, substituted alkyl, alkoxycarbonyl, substituted alkoxycarbonyl, aryl, substituted aryl, arylalkyl, substituted arylalkyl, carbamoyl, cycloalkyl, substituted cycloalkyl, heteroaryl, or substituted heteroaryl. In certain embodiments, R¹ is cyclopropyl, cyclobutyl, cyclopentyl, or cyclohexyl, R² is alkoxycarbonyl or carbamoyl, and R³ is methyl.

In still other embodiments, R¹ is acyl, substituted acyl, alkyl, substituted alkyl, aryl, substituted aryl, arylalkyl, substituted arylalkyl, cycloalkyl, substituted cycloalkyl, cycloheteroalkyl, substituted cycloheteroalkyl, heteroalkyl, substituted heteroalkyl, heteroaryl, substituted heteroaryl, heteroarylalkyl, or substituted heteroarylalkyl, and R² and R³ are independently hydrogen, alkyl, substituted alkyl, aryl, arylalkyl, cycloalkyl, or heteroaryl. In still other embodiments, R¹ is acyl, substituted acyl, alkyl, substituted alkyl, aryl, arylalkyl, cycloalkyl, or heteroaryl, and R² and R³ are independently hydrogen, alkanyl, substituted alkanyl, cycloalkanyl, or substituted cycloalkanyl. In still other embodiments, R¹ is acyl, substituted acyl, alkyl, substituted alkyl, aryl, arylalkyl, cycloalkyl, or heteroaryl, and R² and R³ are independently hydrogen, methyl, ethyl, propyl, isopropyl, butyl, isobutyl, sec-butyl, tert-butyl, cyclopentyl, or cyclohexyl. In the above embodiments, R¹ may be, for example, methyl, ethyl, propyl, isopropyl, butyl, isobutyl, sec-butyl, pentyl, isopentyl, sec-pentyl, neopentyl, 1,1-dimethoxyethyl, 1,1-diethoxyethyl, 1-(1,3-dioxolan-2-yl)-ethyl, 1-(1,3-dioxan-2-yl)-ethyl, 1,1-dimethoxypropyl, 1,1-diethoxypropyl, 1-(1,3-dioxolan-2-yl)-propyl, 1-(1,3-dioxan-2-yl)-propyl, 1,1-dimethoxybutyl, 1,1-diethoxybutyl, 1-(1,3-dioxolan-2-yl)-butyl, 1-(1,3-dioxan-2-yl)-butyl, 1,1-dimethoxybenzyl, 1,1-diethoxybenzyl, 1-(1,3-dioxolan-2-yl)-benzyl, 1-(1,3-dioxan-2-yl)-benzyl, 1,1-dimethoxy-2-phenethyl, 1,1-diethoxy-2-phenethyl, 1-(1,3-dioxolan-2-yl)-2-phenethyl, 1-(1,3-dioxan-2-yl)-2-phenethyl, acetyl, propionyl, butyryl, benzoyl, phenacetyl, phenyl, 4-methoxyphenyl, benzyl, phenethyl, styryl, cyclopropyl, cyclobutyl, cyclopentyl, cyclohexyl, or 3-pyridyl.

In still other embodiments, R¹ is acyl, substituted acyl, alkyl, substituted alkyl, aryl, arylalkyl, cycloalkyl, or heteroaryl, and R² and R³ are independently hydrogen, alkyl, substituted alkyl, aryl, arylalkyl, cycloalkyl, or heteroaryl. In still other embodiments, R¹ is acyl, substituted acyl, alkyl, substituted alkyl, aryl, arylalkyl, cycloalkyl, or heteroaryl, and R² and R³ are independently hydrogen, aryl, arylalkyl, or heteroaryl. In still other embodiments, R¹ is acyl, substituted acyl, alkyl, substituted alkyl, aryl, arylalkyl, cycloalkyl, or heteroaryl, and R² and R³ are independently hydrogen, phenyl, benzyl, phenethyl, or 3-pyridyl. In still other embodiments, R¹ may be, for example, methyl, ethyl, propyl, isopropyl, butyl, isobutyl, sec-butyl, pentyl, isopentyl, sec-pentyl, neopentyl, 1,1-dimethoxyethyl, 1,1-diethoxyethyl, 1-(1,3-dioxolan-2-yl)-ethyl, 1-(1,3-dioxan-2-yl)-ethyl, 1,1-dimethoxypropyl, 1,1-diethoxypropyl, 1-(1,3-dioxolan-2-yl)-propyl, 1-(1,3-dioxan-2-yl)-propyl, 1,1-dimethoxybutyl, 1,1-diethoxybutyl, 1-(1,3-dioxolan-2-yl)-butyl, 1-(1,3-dioxan-2-yl)-butyl, 1,1-dimethoxybenzyl, 1,1-diethoxybenzyl, 1-(1,3-dioxolan-2-yl)-benzyl, 1-(1,3-dioxan-2-yl)-benzyl, 1,1-dimethoxy-2-phenethyl, 1,1-diethoxy-2-phenethyl, 1-(1,3-dioxolan-2-yl)-2-phenethyl, 1-(1,3-dioxan-2-yl)-2-phenethyl, acetyl, propionyl, butyryl, benzoyl, phenacetyl, phenyl, 4-methoxyphenyl, benzyl, phenethyl, styryl, cyclopropyl, cyclobutyl, cyclopentyl, cyclohexyl, or 3-pyridyl.

In still other embodiments, R¹ is acyl, substituted acyl, alkyl, substituted alkyl, aryl, arylalkyl, cycloalkyl, or heteroaryl, and R² and R³ are independently hydrogen, alkyl, substituted alkyl, aryl, arylalkyl, cycloalkyl, or heteroaryl. In still other embodiments, R¹ is acyl, substituted acyl, alkyl, substituted alkyl, aryl, arylalkyl, cycloalkyl, or heteroaryl and R² and R³ are independently hydrogen, alkyl, substituted alkyl, alkoxycarbonyl, substituted alkoxycarbonyl, carbamoyl, or substituted carbamoyl. In certain embodiments, R¹ is acyl, substituted acyl, alkyl, substituted alkyl, aryl, arylalkyl, cycloalkyl, or heteroaryl, R² is alkoxycarbonyl, substituted alkoxycarbonyl, carbamoyl, or substituted carbamoyl, for example methoxycarbonyl, ethoxycarbonyl, propoxycarbonyl, isopropoxycarbonyl, butoxycarbonyl, isobutoxycarbonyl, sec-butoxycarbonyl, tert-butoxycarbonyl, or cyclohexyloxycarbonyl, and R³ is methyl. In the above embodiments, R¹ may be, for example, methyl, ethyl, propyl, isopropyl, butyl, isobutyl, sec-butyl, pentyl, isopentyl, sec-pentyl, neopentyl, 1,1-dimethoxyethyl, 1,1-diethoxyethyl, 1-(1,3-dioxolan-2-yl)-ethyl, 1-(1,3-dioxan-2-yl)-ethyl, 1,1-dimethoxypropyl, 1,1-diethoxypropyl, 1-(1,3-dioxolan 2-yl)-propyl, 1-(1,3-dioxan-2-yl)-propyl, 1,1-dimethoxybutyl, 1,1-diethoxybutyl, 1-(1,3-dioxolan-2-yl)-butyl, 1-(1,3-dioxan-2-yl)-butyl, 1,1-dimethoxybenzyl, 1,1-diethoxybenzyl, 1-(1,3-dioxolan-2-yl)-benzyl, 1-(1,3-dioxan-2-yl)-benzyl, 1,1-dimethoxy-2-phenethyl, 1,1-diethoxy-2-phenethyl, 1-(1,3-dioxolan-2-yl)-2-phenethyl, 1-(1,3-dioxan-2-yl)-2-phenethyl, acetyl, propionyl, butyryl, benzoyl, phenacetyl, phenyl, 4-methoxyphenyl, benzyl, phenethyl, styryl, cyclopropyl, cyclobutyl, cyclopentyl, cyclohexyl, or 3-pyridyl.

In still other embodiments, R¹ is acyl, substituted acyl, alkyl, substituted alkyl, aryl, arylalkyl, cycloalkyl, or heteroaryl, and R² and R³ together with the atom to which they are bonded form a cycloalkyl, substituted cycloalkyl, cycloheteroalkyl, or a substituted cycloheteroalkyl ring. In still other embodiments, R¹ is acyl, substituted acyl, alkyl, substituted alkyl, aryl, arylalkyl, cycloalkyl, or heteroaryl, and R² and R³ together with the atom to which they are bonded form a cycloalkyl or substituted cycloalkyl ring. In still other embodiments, R¹ is acyl, substituted acyl, alkyl, substituted alkyl, aryl, arylalkyl, cycloalkyl, or heteroaryl, and R² and R³ together with the atom to which they are bonded form a cyclobutyl, cyclopentyl, or cyclohexyl ring. In still other embodiments, R¹ may be, for example, methyl, ethyl, propyl, isopropyl, butyl, isobutyl, sec-butyl, pentyl, isopentyl, sec-pentyl, neopentyl, 1,1-dimethoxyethyl, 1,1-diethoxyethyl, 1-(1,3-dioxolan-2-yl)-ethyl, 1-(1,3-dioxan-2-yl)-ethyl, 1,1-dimethoxypropyl, 1,1-diethoxypropyl, 1-(1,3-dioxolan-2-yl)-propyl, 1-(1,3-dioxan-2-yl)-propyl, 1,1-dimethoxybutyl, 1,1-diethoxybutyl, 1-(1,3-dioxolan-2-yl)-butyl, 1-(1,3-dioxan-2-yl)-butyl, 1,1-dimethoxybenzyl, 1,1-diethoxybenzyl, 1-(1,3-dioxolan-2-yl)-benzyl, 1-(1,3-dioxan-2-yl)-benzyl, 1,1-dimethoxy-2-phenethyl, 1,1-diethoxy-2-phenethyl, 1-(1,3-dioxolan-2-yl)-2-phenethyl, 1-(1,3-dioxan-2-yl)-2-phenethyl, acetyl, propionyl, butyryl, benzoyl, phenacetyl, phenyl, 4-methoxyphenyl, benzyl, phenethyl, styryl, cyclopropyl, cyclobutyl, cyclopentyl, cyclohexyl, or 3-pyridyl.

In certain of the above embodiments, R⁴ is hydrogen.

In some embodiments of compounds of Formulae (I) and (II), R⁴ and R³ are each hydrogen, R² is C₁-C₆ alkyl and R¹ is C₁-C₆ alkyl or C₁-C₆ substituted alkyl. In some embodiments, R² is chosen from methyl, ethyl, n-propyl, isopropyl, n-butyl, isobutyl, and sec-butyl, and R¹ is chosen from methyl, ethyl, n-propyl, isopropyl, n-butyl, isobutyl, sec-butyl, n-pentyl, isopentyl, sec-pentyl, neopentyl, and 1,1-diethoxyethyl.

In some embodiments of compounds of Formulae (I) and (II), R² is methyl. In other embodiments of compound of compounds of Formulae (I) and (II), R¹ is methyl, ethyl, n-propyl, or isopropyl. In still other embodiments of compounds of Formulae (I) and (II), R² is methyl and R¹ is methyl, ethyl, n-propyl, or n-butyl. In still other embodiments of compounds of Formulae (I) and (II), R² is ethyl and R¹ is methyl, n-propyl, or isopropyl. In still other embodiments of compounds of Formulae (I) and (II), R² is n-propyl and R¹ is methyl, ethyl, n-propyl, isopropyl, or n-butyl. In still other embodiments of compounds of Formulae (I) and (II), R² is isopropyl and R¹ is methyl, ethyl, n-propyl, isopropyl, n-butyl, or isobutyl. In still other embodiments of compounds of Formulae (I) and (II), R² is n-propyl and R¹ is n-propyl. In still other embodiments of compounds of Formulae (I) and (II), R² is methyl and R¹ is ethyl. In still other embodiments of compounds of Formulae (I) and (II), R² is methyl and R¹ is isopropyl. In still other embodiments of compounds of Formulae (I) and (II), R² is isopropyl and R¹ is isopropyl. In still other embodiments of compounds of Formulae (I) and (II), R² is isopropyl and R¹ is 1,1-diethoxyethyl. In still other embodiments of compounds of Formulae (I) and (II), R² is propyl and R¹ is isopropyl. In still other embodiments of compounds of Formulae (I) and (II), R² is propyl and R1 is ethyl.

In some embodiments, the compound of Formula (I) where R¹ is isopropyl, R² is methyl and R³ is hydrogen is a crystalline form of 1-{[(α-isobutanoyloxyethoxy)carbonyl]aminomethyl}-1-cyclohexane acetic acid as disclosed in Estrada et al., WO 2005/037784.

Suitable examples of Formula (I) compounds include 1-{[(α-acetoxyethoxy)carbonyl]aminomethyl}-1-cyclohexane acetic acid, 1-{[(α-propanoyloxyethoxy)carbonyl]aminomethyl}-1-cyclohexane acetic acid, 1-{[(α-butanoyloxyethoxy)carbonyl]aminomethyl}-1-cyclohexane acetic acid, 1-{[(α-isobutanoyloxyethoxy)carbonyl]aminomethyl}-1-cyclohexane acetic acid, 1-{[(α-pivaloxyethoxy)carbonyl]aminomethyl}-1-cyclohexane acetic acid, 1-{[(α-acetoxymethoxy)carbonyl]aminomethyl}-1-cyclohexane acetic acid, 1-{[(α-propanoyloxymethoxy)carbonyl]aminomethyl}-1-cyclohexane acetic acid, 1-{[(α-butanoyloxymethoxy)carbonyl]aminomethyl}-1-cyclohexane acetic acid, 1-{[(α-isobutanoyloxymethoxy)carbonyl]aminomethyl}-1-cyclohexane acetic acid, 1-{[(α-pivaloxymethoxy)carbonyl]aminomethyl}-1-cyclohexane acetic acid, 1-{[(α-acetoxypropoxy)carbonyl]aminomethyl}-1-cyclohexane acetic acid, 1-{[(α-propanoyloxypropoxy)carbonyl]aminomethyl}-1-cyclohexane acetic acid, 1-{[(α-butanoyloxypropoxy)carbonyl]aminomethyl}-1-cyclohexane acetic acid, 1-{[(α-isobutanoyloxypropoxy)carbonyl]aminomethyl}-1-cyclohexane acetic acid, 1-{[(α-pivaloxypropoxy)carbonyl]aminomethyl}-1-cyclohexane acetic acid, 1-{[(α-acetoxyisopropoxy)carbonyl]aminomethyl}-1-cyclohexane acetic acid, 1-{[(α-propanoyloxyisopropoxy)carbonyl]aminomethyl}-1-cyclohexane acetic acid, 1-{[(α-butanoyloxyisopropoxy)carbonyl]aminomethyl}-1-cyclohexane acetic acid, 1-{[(α-isobutanoyloxyisopropoxy)carbonyl]aminomethyl}-1-cyclohexane acetic acid, 1-{[(α-pivaloxyisopropoxy)carbonyl]aminomethyl}-1-cyclohexane acetic acid, 1-{[(α-acetoxybutoxy)carbonyl]aminomethyl}-1-cyclohexane acetic acid, 1-{[(α-propanoyloxybutoxy)carbonyl]aminomethyl}-1-cyclohexane acetic acid, 1-{[(α-butanoyloxybutoxy)carbonyl]aminomethyl}-1-cyclohexane acetic acid, 1-{[(α-isobutanoyloxybutoxy)carbonyl]aminomethyl}-1-cyclohexane acetic acid, and 1-{[(α-pivaloxybutoxy)carbonyl]aminomethyl}-1-cyclohexane acetic acid, or pharmaceutically acceptable salts thereof, pharmaceutically acceptable solvates of any of the foregoing, or pharmaceutically acceptable N-oxides of any of the foregoing.

Suitable examples of Formula (II) compounds include 3-{[(α-acetoxyethoxy)carbonyl]aminomethyl}-5-methyl hexanoic acid, 3-{[(α-propanoyloxyethoxy)carbonyl]aminomethyl}-5-methyl hexanoic acid, 3-{[(α-butanoyloxyethoxy)carbonyl]aminomethyl}-5-methyl hexanoic acid, 3-{[(α-isobutanoyloxyethoxy)carbonyl]aminomethyl}-5-methyl hexanoic acid, 3-{[(α-pivaloxyethoxy)carbonyl]aminomethyl}-5-methyl hexanoic acid, 3-{[(α-acetoxymethoxy)carbonyl]aminomethyl}-5-methyl hexanoic acid, 3-{[(α-propanoyloxymethoxy)carbonyl]aminomethyl}-5-methyl hexanoic acid, 3-{[(α-butanoyloxymethoxy)carbonyl]aminomethyl}-5-methyl hexanoic acid, 3-{[(α-isobutanoyloxymethoxy)carbonyl]aminomethyl}-5-methyl hexanoic acid, 3-{[(α-pivaloxymethoxy)carbonyl]aminomethyl}-5-methyl hexanoic acid, 3-{[(α-acetoxypropoxy)carbonyl]aminomethyl}-5-methyl hexanoic acid, 3-{[(α-propanoyloxypropoxy)carbonyl]aminomethyl}-5-methyl hexanoic acid, 3-{[(α-butanoyloxypropoxy)carbonyl]aminomethyl}-5-methyl hexanoic acid, 3-{[(α-isobutanoyloxypropoxy)carbonyl]aminomethyl}-5-methyl hexanoic acid, 3-{[(α-pivaloxypropoxy)carbonyl]aminomethyl}-5-methyl hexanoic acid, 3-{[(α-acetoxyisopropoxy)carbonyl]aminomethyl}-5-methyl hexanoic acid, 3-{[(α-propanoyloxyisopropoxy)carbonyl]aminomethyl}-5-methyl hexanoic acid, 3-{[(α-butanoyloxyisopropoxy)carbonyl]aminomethyl}-5-methyl hexanoic acid, 3-{[(α-isobutanoyloxyisopropoxy)carbonyl]aminomethyl}-5-methyl hexanoic acid, 3-{[(α-pivaloxyisopropoxy)carbonyl]aminomethyl}-5-methyl hexanoic acid, 3-{[(α-acetoxybutoxy)carbonyl]aminomethyl}-5-methyl hexanoic acid, 3-{[(α-propanoyloxybutoxy)carbonyl]aminomethyl}-5-methyl hexanoic acid, 3-{[(α-butanoyloxybutoxy)carbonyl]aminomethyl}-5-methyl hexanoic acid, 3-{[(α-isobutanoyloxybutoxy)carbonyl]aminomethyl}-5-methyl hexanoic acid, and 3-{[(α-pivaloxybutoxy)carbonyl]aminomethyl}-5-methyl hexanoic acid, or pharmaceutically acceptable salts thereof, pharmaceutically acceptable solvates of any of the foregoing, or pharmaceutically acceptable N-oxides of any of the foregoing.

### 4.3 Methods of Synthesis of Prodrugs of Gabapentin and Pregabalin

Methods of synthesis of prodrugs of gabapentin and pregabalin, including methods of synthesizing compounds of structural Formulae (I) and (II) are disclosed in Gallop et al., U.S. Pat. No. 6,818,787, Gallop et al., U.S. Patent Publication No. 2004/0077553 and Bhat et al., U.S. Patent Publication No. 2005/0070715, each of which is incorporated herein by reference. Other methods for synthesis of prodrugs of gabapentin and pregabalin have also been disclosed (see Bryans et al., WO 01/90052; U.K. Application GB 2,362,646; European Applications EP 1,201,240 and 1,178,034; Yatvin et al., U.S. Pat. No. 6,024,977; Gallop et al., U.S. Patent Publication No. 2002/0151529; Gallop et al., U.S. Patent Publication No. 2002/0142998; Gallop et al., U.S. Patent Publication No. 2002/0098999; Cundy et al., U.S. Pat. No. 6,900,192; and Gallop et al., WO 02/42414).

### 4.4 Therapeutic Methods of Use

In some embodiments, a prodrug of a gabapentin or pregabalin, and/or pharmaceutical composition thereof, is administered to a male patient suffering from premature ejaculation. The suitability of gabapentin and pregabalin prodrugs and/or pharmaceutical compositions thereof to treat premature ejaculation may be readily determined by methods known to the skilled artisan.

Administering a gabapentin or pregabalin prodrug, or a pharmaceutical composition thereof, to a male patient suffering from premature ejaculation increases the ejaculatory latency (i.e., the time between first penetration and ejaculation) of the male patient. Increased ejaculatory latency can be measured objectively, using a clock or a stopwatch.

The compounds disclosed herein, for example, the gabapentin prodrug 1-{[(α-isobutanoyloxyethoxy)carbonyl]aminomethyl}-1-cyclohexane acetic acid, is more efficacious than the parent drug molecule (i.e., gabapentin) in treating premature ejaculation because the disclosed compounds require less time to reach a therapeutic concentration in the blood, i.e., the compounds disclosed herein have a shorter Tₘₐₓ than their gabapentin or pregabalin “counterparts” when taken orally. For example, an immediate release formulation of the prodrug 1-{[(α-isobutanoyloxyethoxy)carbonyl]aminomethyl}-1-cyclohexane acetic acid provides a Tₘₐₓ within from about 2 hours to about 2.6 hours following oral administration compared from about 2.8 to about 3.3 hours for an equivalent immediate release formulation of the parent drug, gabapentin. This provides an advantage over the parent drugs (i.e., gabapentin and pregabalin) in that the patient need not wait as long after administering the prodrugs before a therapeutic concentration of the active parent drug in the blood is reached. Thus, the prodrugs disclosed herein allow the patient to dose shortly before sexual activity, allowing sexual activity to occur with more spontaneity, compared to dosing of either gabapentin or pregabalin.

Another potential advantage of the prodrugs disclosed herein compared to either gabapentin or pregabalin is that the prodrugs are capable of being absorbed from the colon while the parent drugs are not. This allows the prodrugs to be formulated as an oral sustained release formulation, which provides for sustained release over a period of hours into the gastrointestinal tract, for example, within the colon. This means that the compounds, for example the gabapentin prodrug 1-{[(α-isobutanoyloxyethoxy)carbonyl]aminomethyl}-1-cyclohexane acetic acid), can be dosed less frequently for maintenance of a therapeutically effective drug concentration in the blood. For example, the gabapentin and pregabalin prodrugs disclosed herein can be formulated as an oral sustained release dosage form that can be taken shortly before sexual activity and provides a therapeutic window of at least about 4 to 12 hours, and in some cases up to about 20 hours, during which sexual activity can occur.

### 4.5 Therapeutic/Prophylactic Administration

Dosage forms comprising prodrugs of gabapentin and pregabalin may be used to treat premature ejaculation. The dosage forms may be administered or applied singly, or in combination with one or more other pharmaceutically active agents. The dosage forms may also deliver a prodrug of gabapentin or pregabalin to a male patient in combination with another pharmaceutically active agent, including one or more of the following drugs: apomorphine; dopamine receptor antagonists, for example, dopamine D2, D3, and D4 antagonists such as Premiprixal, Pharmacia Upjohn compound number PNU95666 or levosulphiride; serotonin receptor antagonist or modulator, for example, antagonists or modulators for 5HT1A, including NAD-299 (robalzotan) and WAY-100635, and/or for example, antagonists or modulators for 5HT3 receptors, including batanopirde, granisetron, ondansetron, tropistron, and MDL-73147EF; serotonin receptor agonist or modulator, for example, agonists or modulators for 5HT2C, 5HT1B and/or 5HT1D receptors, including anpirtoline, sumatriptan, eletriptan, frovatriptan, and other triptans well known as anti-migraine medication; alpha-adrenergic receptor antagonist (also known as alpha-adrenergic blockers, alpha-blockers or alpha-receptor blockers) including phentolamine, prazosin, phentolamine mesylate, trazodone, alfuzosin, indoramin, naftopidil, tamsulosin, phenoxybenzamine, rauwolfa alkaloids, Recordati 15/2739, SNAP 1069, SNAP 5089, RS17053, SL 89.0591, doxazosin, terazosin, and abanoquil; alpha-2-adrenergic receptor antagonists including dibenamine, tolazoline, trimazosin, efaroxan, yohimbine, idazoxan clonidine, and dibenamine; non-selective alpha-adrenergic receptor antagonists including dapiprazole; oxytocin receptor antagonists such as L-368 899 (the synthesis of which is taught in Williams et al., J. Med. Chem. 37: 565-571 (1994)); and vasopressin receptor antagonists.

When used in the present methods of treatment, upon releasing a prodrug of gabapentin or pregabalin in vivo, the dosage forms can provide gabapentin or pregabalin in the systemic circulation of the male patient. While not wishing to bound by theory, the promoiety or promoieties of the prodrug may be cleaved either chemically and/or enzymatically. One or more enzymes present in the stomach, intestinal lumen, intestinal tissue, blood, liver, brain or any other suitable tissue of a mammal may cleave the promoiety or promoieties of the prodrug. The mechanism of cleavage is not critical for the current methods. In certain embodiments, the gabapentin or pregabalin that is formed by cleavage of the promoiety from the prodrug does not contain substantial quantities of lactam contaminant (such as, less than about 0.5% by weight, for example, less than about 0.2% by weight, and in certain embodiments, less than about 0.1% by weight) for the reasons described in Augart et al., U.S. Pat. No. 6,054,482. The extent of release of lactam contaminant from the prodrugs may be assessed using standard in vitro analytical methods.

### 4.6 Pharmaceutical Compositions

Pharmaceutical compositions disclosed herein comprise a therapeutically effective amount of at least one gabapentin and/or pregabalin prodrug, together with a suitable amount of a pharmaceutically acceptable vehicle, so as to provide a form for proper administration to a male patient. In certain embodiments, the one or more gabapentin and/or pregabalin prodrug is in a purified form. When administered to a male patient, the prodrug and pharmaceutically acceptable vehicles may be sterile. Suitable pharmaceutical vehicles include excipients such as starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol, and the like. The present pharmaceutical compositions, if desired, can also contain minor amounts of wetting or emulsifying agents, or pH buffering agents. In addition, auxiliary, stabilizing, thickening, lubricating, and coloring agents may be used.

Pharmaceutical compositions may be manufactured by means of conventional mixing, dissolving, granulating, dragee-making, levigating, emulsifying, encapsulating, entrapping, or lyophilizing processes. Pharmaceutical compositions may be formulated in conventional manner using one or more physiologically acceptable carriers, diluents, excipients, or auxiliaries, which facilitate processing of compounds disclosed herein into preparations, which can be used pharmaceutically. Proper formulation is dependent upon the route of administration chosen.

The present pharmaceutical compositions can take the form of solutions, suspensions, emulsion, tablets, pills, pellets, capsules, capsules containing liquids, powders, sustained-release formulations, suppositories, emulsions, aerosols, sprays, suspensions, or any other form suitable for use. In some embodiments, the pharmaceutically acceptable vehicle is a capsule (see e.g., Grosswald et al., U.S. Pat. No. 5,698,155). Other examples of suitable pharmaceutical vehicles have been described in the art (see Remington's Pharmaceutical Sciences, Philadelphia College of Pharmacy and Science, 19th Edition, 1995). In certain embodiments, pharmaceutical compositions can be formulated for oral delivery, for example, for oral sustained release administration.

Pharmaceutical compositions for oral delivery may be in the form of tablets, lozenges, aqueous or oily suspensions, granules, powders, emulsions, capsules, syrups, or elixirs, for example. Orally administered compositions may contain one or more optional agents, for example, sweetening agents such as fructose, aspartame or saccharin, flavoring agents such as peppermint, oil of wintergreen, or cherry coloring agents and preserving agents, to provide a pharmaceutically palatable preparation. Moreover, when in tablet or pill form, the compositions may be coated to delay disintegration and absorption in the gastrointestinal tract, thereby providing a sustained action over an extended period of time. Oral compositions can include standard vehicles such as mannitol, lactose, starch, magnesium stearate, sodium saccharine, cellulose, magnesium carbonate, etc. Such vehicles may be of pharmaceutical grade.

For oral liquid preparations such as, for example, suspensions, elixirs and solutions, suitable carriers, excipients, or diluents include water, saline, alkyleneglycols (e.g., propylene glycol), polyalkylene glycols (e.g., polyethylene glycol) oils, alcohols, slightly acidic buffers ranging from about pH 4 to about pH 6 (e.g., acetate, citrate, ascorbate ranging from about 5 mM to about 50 mM), etc. Additionally, flavoring agents, preservatives, coloring agents, bile salts, acylcarnitines, and the like may be added.

When a gabapentin or pregabalin prodrug is acidic, it may be included in any of the above-described formulations as the free acid, a pharmaceutically acceptable salt, or a solvate. Pharmaceutically acceptable salts substantially retain the activity of the free acid, may be prepared by reaction with bases and tend to be more soluble in aqueous and other protic solvents than the corresponding free acid form.

In certain embodiments, pharmaceutical compositions of the present disclosure contain no or only low levels of lactam side products formed by intramolecular cyclization of the parent gabapentin or pregabalin compound as well as the prodrug itself. In certain embodiments, the compositions are stable to extended storage (for example, greater than one year) without substantial lactam formation (for example, less than about 0.5% lactam by weight, such as, less than about 0.2% lactam by weight, and in certain embodiments, less than about 0.1% lactam by weight).

Pharmaceutical compositions provided by the present disclosure may be provided as immediate release or sustained release formulations.

### 4.7 Sustained Release Oral Dosage Forms

The methods that involve oral administration of a gabapentin or pregabalin prodrug to treat premature ejaculation can be practiced with a number of different dosage forms, which provide sustained release of the prodrug. Such sustained release oral dosage forms may be used for administering the gabapentin and pregabalin prodrugs of Formulae (I) and (II) since those prodrugs are absorbed by cells lining the large intestine, and because these dosage forms are generally well adapted to deliver a prodrug to that location of the gastrointestinal tract.

In some embodiments, the dosage form is comprised of beads that on dissolution or diffusion release the prodrug over an extended period of hours, for example, over a period of at least about 6 hours, such as over a period of at least about 8 hours and in certain embodiments, over a period of at least about 12 hours. The prodrug-releasing beads may have a central composition or core comprising a prodrug and pharmaceutically acceptable vehicles, including optional lubricants, antioxidants and buffers. The beads may be medical preparations with a diameter of about 1 to about 2 mm. Individual beads may comprise doses of the prodrug, for example, doses of up to about 40 mg of prodrug. The beads, in some embodiments, are formed of non-cross-linked materials to enhance their discharge from the gastrointestinal tract. The beads may be coated with a release rate-controlling polymer that gives a timed-release profile.

The time-release beads may be manufactured into a tablet for therapeutically effective prodrug administration. The beads can be made into matrix tablets by direct compression of a plurality of beads coated with, for example, an acrylic resin and blended with excipients such as hydroxypropylmethyl cellulose. The manufacture of beads has been disclosed in the art (Lu, Int. J. Pharm., 112: 117-124 (1994); Pharmaceutical Sciences by Remington, 14th ed, pp 1626-1628 (1970); Fincher, J. Pharm. Sci., 57, 1825-1835 (1968); Benedikt, U.S. Pat. No. 4,083,949), as has the manufacture of tablets (Pharmaceutical Sciences, by Remington, 17th Ed, Ch. 90, pp 1603-1625 (1985)).

In other embodiments, an oral sustained release pump may be used (Langer, supra; Sefton, CRC Crit Ref Biomed. Eng., 14: 201 (1987); Saudek et al., N. Engl. J. Med., 321: 574 (1989)).

In other embodiments, polymeric materials can be used (See “Medical Applications of Controlled Release,” Langer and Wise (eds.), CRC Press., Boca Raton, Fla. (1974); “Controlled Drug Bioavailability,” Drug Product Design and Performance, Smolen and Ball (eds.), Wiley, New York (1984); Langer et al., J Macromol. Sci. Rev. Macromol Chem., 23: 61 (1983); Levy et al., Science 228: 190 (1985); During et al., Ann. Neurol., 25: 351 (1989); Howard et al., J. Neurosurg., 71:105 (1989)). In certain embodiments, polymeric materials are used for oral sustained release delivery. Examples of polymers for oral sustained release delivery include sodium carboxymethylcellulose, hydroxypropylcellulose, hydroxypropyl methylcellulose and hydroxyethylcellulose, such as hydroxypropylmethylcellulose. Other examples of cellulose ethers have been described (Alderman, Int. J. Pharm. Tech. & Prod. Mfr., 5(3): 1-9 (1984)). Factors affecting drug release are well known to the skilled artisan and have been described in the art (Bamba et al., Int. J. Pharm., 2: 307 (1979)).

In other embodiments, enteric-coated preparations can be used for oral sustained release administration. Examples of coating materials include polymers with a pH-dependent solubility (i.e., pH-controlled release), polymers with a slow or pH-dependent rate of swelling, dissolution or erosion (i.e., time-controlled release), polymers that are degraded by enzymes (i.e., enzyme-controlled release) and polymers that form firm layers that are destroyed by an increase in pressure (i.e., pressure-controlled release).

In yet other embodiments, drug-releasing lipid matrices can be used for oral sustained release administration. One example is when solid microparticles of the prodrug are coated with a thin controlled release layer of a lipid (e.g., glyceryl behenate and/or glyceryl palmitostearate) as disclosed in Farah et al., U.S. Pat. No. 6,375,987 and Joachim et al., U.S. Pat. No. 6,379,700. The lipid-coated particles can optionally be compressed to form a tablet. Another controlled release lipid-based matrix material, which is suitable for sustained release oral administration, comprises polyglycolized glycerides as disclosed in Roussin et al., U.S. Pat. No. 6,171,615.

In yet other embodiments, prodrug-releasing waxes can be used for oral sustained release administration. Examples of suitable sustained prodrug-releasing waxes are disclosed in Cain et al., U.S. Pat. No. 3,402,240 (carnauba wax, candelilla wax, esparto wax and ouricury wax); Shtohryn et al., U.S. Pat. No. 4,820,523 (hydrogenated vegetable oil, bees wax, caranuba wax, paraffin, candelilla, ozokerite, and mixtures thereof); and Walters, U.S. Pat. No. 4,421,736 (mixture of paraffin and castor wax).

In still other embodiments, osmotic delivery systems are used for oral sustained release administration (Verma et al., Drug Dev. Ind. Pharm., 26: 695-708 (2000)). In certain embodiments, OROS® systems made by Alza Corporation, Mountain View, Calif. are used for oral sustained release delivery devices (Theeuwes et al., U.S. Pat. No. 3,845,770; Theeuwes et al., U.S. Pat. No. 3,916,899).

In yet other embodiments, a controlled-release system can be placed in proximity of the target of the prodrug of gabapentin or pregabalin, thus requiring only a fraction of the systemic dose (See, e.g., Goodson, in “Medical Applications of Controlled Release,” supra, vol. 2, pp. 115-138 (1984)). Other controlled-release systems are discussed in Langer, Science, 249:1527-1533 (1990), may also be used.

In other embodiments, the dosage form comprises a prodrug of gabapentin or pregabalin coated on a polymer substrate. The polymer can be an erodible, or a nonerodible polymer. The coated substrate may be folded onto itself to provide a bilayer polymer drug dosage form. For example, a prodrug of gabapentin or pregabalin can be coated onto a polymer such as a polypeptide, collagen, gelatin, polyvinyl alcohol, polyorthoester, polyacetyl, or a polyorthocarbonate and the coated polymer folded onto itself to provide a bilaminated dosage form. In operation, the bioerodible dosage form erodes at a controlled rate to dispense the prodrug over a sustained release period. Representative biodegradable polymers comprise a member chosen from biodegradable poly(amides), poly(amino acids), poly(esters), poly(lactic acid), poly(glycolic acid), poly(carbohydrate), poly(orthoester), poly (orthocarbonate), poly(acetyl), poly(anhydrides), biodegradable poly(dihydropyrans), and poly(dioxinones) which are known in the art (Rosoff, Controlled Release of Drugs, Chap. 2, pp. 53-95 (1989); Heller et al., U.S. Pat. No. 3,811,444; Michaels, U.S. Pat. No. 3,962,414; Capozza, U.S. Pat. No. 4,066,747; Schmitt, U.S. Pat. No. 4,070,347; Choi et al., U.S. Pat. No. 4,079,038; Choi et al., U.S. Pat. No. 4,093,709).

In other embodiments, the dosage form comprises a prodrug loaded into a polymer, such as a prodrug-releasing polymer, that releases the prodrug by diffusion through a polymer, or by flux through pores or by rupture of a polymer matrix. The drug delivery polymeric dosage form comprises a concentration of about 10 mg to about 2500 mg homogenously contained in or on a polymer. The dosage form comprises at least one exposed surface at the beginning of dose delivery. The non-exposed surface, when present, is coated with a pharmaceutically acceptable material impermeable to the passage of a prodrug. The dosage form may be manufactured by procedures known in the art. An example of providing a dosage form comprises blending a pharmaceutically acceptable carrier like polyethylene glycol, with a known dose of prodrug at an elevated temperature, (e.g., about 37° C.), and adding it to a silastic medical grade elastomer with a cross-linking agent, for example, octanoate, followed by casting in a mold. The step is repeated for each optional successive layer. The system is allowed to set for about 1 hour, to provide the dosage form. Representative polymers for manufacturing the dosage form comprise a member chosen from olefin, and vinyl polymers, addition polymers, condensation polymers, carbohydrate polymers, and silicone polymers as represented by polyethylene, polypropylene, polyvinyl acetate, polymethylacrylate, polyisobutylmethacrylate, poly alginate, polyamide and polysilicone. The polymers and procedures for manufacturing them have been described in the art (Coleman et al., Polymers, 31: 1187-1231 (1990); Roerdink et al., Drug Carrier Systems, 9: 57-10 (1989); Leong et al., Adv. Drug Delivery Rev., 1: 199-233 (1987); Roff et al., Handbook of Common Polymers 1971, CRC Press; Chien et al., U.S. Pat. No. 3,992,518).

In other embodiments, the dosage from comprises a plurality of pills. The time-release pills provide a number of individual doses for providing various time doses for achieving a sustained-release prodrug delivery profile over an extended period of time up to about 24 hours. The matrix comprises a hydrophilic polymer chosen from a polysaccharide, agar, agarose, natural gum, alkali alginate including sodium alginate, carrageenan, fucoidan, furcellaran, laminaran, hypnea, gum arabic, gum ghatti, gum karaya, gum tragacanth, locust bean gum, pectin, amylopectin, gelatin, and a hydrophilic colloid. The hydrophilic matrix comprises a plurality of from about 4 to about 50 pills, each pill comprise a dose population of from about 10 ng, about 0.5 mg, about 1 mg, about 1.2 mg, about 1.4 mg, about 1.6 mg, about 5.0 mg, etc. The pills comprise a release rate-controlling wall of from about 0.001 mm to about 10 mm thickness to provide for the timed release of prodrug. Representative wall forming materials include a triglyceryl ester chosen from glyceryl tristearate, glyceryl monostearate, glyceryl dipalmitate, glyceryl laureate, glyceryl didecenoate and glyceryl tridenoate. Other wall forming materials comprise polyvinyl acetate, phthalate, methylcellulose phthalate and microporous olefins. Procedures for manufacturing pills are disclosed in Urquhart et al., U.S. Pat. No. 4,434,153; Urquhart et al., U.S. Pat. No. 4,721,613; Theeuwes, U.S. Pat. No. 4,853,229; Barry, U.S. Patent No. 2,996,431; Neville, U.S. Pat. No. 3,139,383; Mehta, U.S. Pat. No. 4,752,470.

In other embodiments, the dosage form comprises an osmotic dosage form, which comprises a semipermeable wall that surrounds a therapeutic composition comprising the prodrug. In use within a male patient, the osmotic dosage form comprising a homogenous composition, imbibes fluid through the semipermeable wall into the dosage form in response to the concentration gradient across the semipermeable wall. The therapeutic composition in the dosage form develops osmotic pressure differential that causes the therapeutic composition to be administered through an exit from the dosage form over a prolonged period of time up to about 24 hours (or even in some cases up to about 30 hours) to provide controlled and sustained prodrug release. These delivery platforms can provide a zero order, or an essentially zero order, delivery profile, as opposed to the spiked profiles of immediate release formulations.

In other embodiments, the dosage form comprises another osmotic dosage form comprising a wall surrounding a compartment, the wall comprising a semipermeable polymeric composition permeable to the passage of fluid and substantially impermeable to the passage of prodrug present in the compartment, a prodrug-containing layer composition in the compartment, a hydrogel push layer composition in the compartment comprising an osmotic formulation for imbibing and absorbing fluid for expanding in size for pushing the prodrug composition layer from the dosage form, and at least one passageway in the wall for releasing the prodrug composition. The method delivers the prodrug by imbibing fluid through the semipermeable wall at a fluid imbibing rate determined by the permeability of the semipermeable wall and the osmotic pressure across the semipermeable wall causing the push layer to expand, thereby delivering the prodrug from the dosage form through the exit passageway to a male patient over a prolonged period of time (up to about 24 or even up to about 30 hours). The hydrogel layer composition may comprise from about 10 mg to about 1000 mg of a hydrogel such as a member chosen from a polyalkylene oxide of from about 1,000,000 to 8,000,000 weight-average molecular weight which are chosen from a polyethylene oxide of about 1,000,000 weight-average molecular weight, a polyethylene oxide of about 2,000,000 weight-average molecular weight, a polyethylene oxide of about 4,000,000 weight-average molecular weight, a polyethylene oxide of about 5,000,000 weight-average molecular weight, a polyethylene oxide of about 7,000,000 weight-average molecular weight and a polypropylene oxide ranging from about 1,000,000 to about 8,000,000 weight-average molecular weight; or ranging from about 10 mg to about 1000 mg of an alkali carboxymethylcellulose ranging from about 10,000 to about 6,000,000 weight-average molecular weight, such as sodium carboxymethylcellulose or potassium carboxymethylcellulose. The hydrogel expansion layer comprises from about 0.0 mg to about 350 mg, in present manufacture; from about 0.1 mg to about 250 mg of a hydroxyalkylcellulose ranging from about 7,500 to about 4,500,00 weight-average molecular weight (e.g., hydroxymethylcellulose, hydroxyethylcellulose, hydroxypropylcellulose, hydroxybutylcellulose or hydroxypentylcellulose) in present manufacture; ranging from about 1 mg to about 50 mg of an osmagent chosen from sodium chloride, potassium chloride, potassium acid phosphate, tartaric acid, citric acid, raffinose, magnesium sulfate, magnesium chloride, urea, inositol, sucrose, glucose and sorbitol; from about 0 to about 5 mg of a colorant, such as ferric oxide; from about 0 mg to about 30 mg, in a present manufacture, from about 0.1 mg to about 30 mg of a hydroxypropylalkylcellulose ranging from about 9,000 to about 225,000 average-number molecular weight, chosen from hydroxypropylethylcellulose, hydroxypropyl pentylcellulose, hydroxypropylmethylcellulose, and hydropropylbutylcellulose; from about 0.00 to about 1.5 mg of an antioxidant chosen from ascorbic acid, butylated hydroxyanisole, butylated hydroxyquinone, butylhydroxyanisole, hydroxycomarin, butylated hydroxytoluene, cephalm, ethyl gallate, propyl gallate, octyl gallate, lauryl gallate, propyl-hydroxybenzoate, trihydroxybutylrophenone, dimethylphenol, dibutylphenol, vitamin E, lecithin and ethanolamine; and from about 0.0 mg to about 7 mg of a lubricant chosen from calcium stearate, magnesium stearate, zinc stearate, magnesium oleate, calcium palmitate, sodium suberate, potassium laurate, salts of fatty acids, salts of alicyclic acids, salts of aromatic acids, stearic acid, oleic acid, palmitic acid, a mixture of a salt of a fatty, alicyclic or aromatic acid, and a fatty, alicyclic, or aromatic acid.

In the osmotic dosage forms, the semipermeable wall comprises a composition that is permeable to the passage of fluid and impermeable to the passage of prodrug. The wall is nontoxic and comprises a polymer chosen from cellulose acylate, cellulose diacylate, cellulose triacylate, cellulose acetate, cellulose diacetate and cellulose triacetate. The wall comprises about 75 wt % (weight percent) to about 100 wt % of the cellulosic wall-forming polymer; or, the wall can comprise additionally about 0.01 wt % to about 80 wt % of polyethylene glycol, or about 1 wt % to about 25 wt % of a cellulose ether chosen from hydroxypropylcellulose or a hydroxypropylalkycellulose such as hydroxypropylmethylcellulose. The total weight percent of all components comprising the wall is equal to about 100 wt %. The internal compartment comprises the prodrug-containing composition alone or in layered position with an expandable hydrogel composition. The expandable hydrogel composition in the compartment increases in dimension by imbibing the fluid through the semipermeable wall, causing the hydrogel to expand and occupy space in the compartment, whereby the drug composition is pushed from the dosage form. The therapeutic layer and the expandable layer act together during the operation of the dosage form for the release of prodrug to a male patient over time. The dosage form comprises a passageway in the wall that connects the exterior of the dosage form with the internal compartment. The osmotic powered dosage form can be made to deliver prodrug from the dosage form to the male patient at a zero order rate of release over a period of up to about 24 hours.

The expression “passageway” as used herein comprises means and methods suitable for the metered release of the prodrug from the compartment of the dosage form. The exit means comprises at least one passageway, including orifice, bore, aperture, pore, porous element, hollow fiber, capillary tube, channel, porous overlay, or porous element that provides for the osmotic controlled release of prodrug. The passageway includes a material that erodes or is leached from the wall in a fluid environment of use to produce at least one controlled-release dimensioned passageway. Representative materials suitable for forming a passageway, or a multiplicity of passageways comprise a leachable poly(glycolic) acid or poly(lactic) acid polymer in the wall, a gelatinous filament, poly(vinyl alcohol), leach-able polysaccharides, salts, and oxides. A pore passageway, or more than one pore passageway, can be formed by leaching a leachable compound, such as sorbitol, from the wall. The passageway possesses controlled-release dimensions, such as round, triangular, square and elliptical, for the metered release of prodrug from the dosage form. The dosage form can be constructed with one or more passageways in spaced apart relationship on a single surface or on more than one surface of the wall. The expression “fluid environment” denotes an aqueous or biological fluid as in a human male patient, including the gastrointestinal tract. Passageways and equipment for forming passageways are disclosed in Theeuwes et al., U.S. Pat. No. 3,845,770; Theeuwes et al., U.S. Pat. No. 3,916,899; Saunders et al., U.S. Pat. No. 4,063,064; Theeuwes et al., U.S. Pat. No. 4,088,864 and Ayer et al., U.S. Pat. No. 4,816,263. Passageways formed by leaching are disclosed in Ayer et al., U.S. Pat. No. 4,200,098, and Ayer et al., U.S. Pat. No. 4,285,987.

Regardless of the specific form of sustained release oral dosage form used, the prodrug can be released from the dosage form over a period of at least about 4 hours, for example, over a period of at least about 8 hours, and in certain embodiments, over a period of at least about 12 hours. The sustained release oral dosage form further provides a concentration of gabapentin or pregabalin in the blood plasma of the male patient over time, which curve has an area under the curve (AUC) that is proportional to the dose of the prodrug of gabapentin or pregabalin administered, and a maximum concentration Cₘₐₓ. The Cₘₐₓ can be less than 75%, for example, less than 60%, of the Cₘₐₓ obtained from administering an equivalent dose of the prodrug from an immediate release oral dosage form, and the AUC is substantially the same as the AUC obtained from administering an equivalent dose of the prodrug from an immediate release oral dosage form.

### 4.8 Methods of Administration and Doses

The present methods for treatment of premature ejaculation require administration of a gabapentin or pregabalin prodrug, or a pharmaceutical composition thereof, to a male patient in need of such treatment. The compounds and/or pharmaceutical compositions thereof can be administered orally. The compounds and/or pharmaceutical compositions thereof may also be administered by any other convenient route, for example, by infusion or bolus injection, by absorption through epithelial or mucocutaneous linings (e.g., oral mucosa, rectal and intestinal mucosa, etc.). Administration can be systemic or local. Various delivery systems are known, (e.g., encapsulation in liposomes, microparticles, microcapsules, capsules, etc.) that can be used to administer a compound and/or pharmaceutical composition thereof. Methods of administration include, but are not limited to, intradermal, intramuscular, intraperitoneal, intravenous, subcutaneous, intranasal, epidural, oral, sublingual, intranasal, intracerebral, intravaginal, transdermal, rectally, by inhalation, or topically, for example, to the ears, nose, eyes, or skin. In certain embodiments, the compounds and/or pharmaceutical compositions thereof are delivered via sustained release dosage forms, for example via oral sustained release dosage forms.

The amount of gabapentin or pregabalin prodrug that will be effective in the treatment of premature ejaculation in a male patient will depend on a number of factors including the weight of the patient, the sensitivity of the patient to gabapentin or pregabalin, and the manner and route of administration of the prodrug. The therapeutic dose can be determined by standard clinical techniques known in the art. In addition, in vitro or in vivo assays may optionally be employed to help identify optimal dosage ranges.

In certain embodiments, the dosage forms are adapted to be administered to a male patient on an as needed basis. For those patients engaging in sexual activity on a daily basis, the dosage forms are adapted to be administered no more than twice per day, for example, only once per day. Dosing may be provided alone or in combination with other drugs and may continue as long as required for effective treatment of premature ejaculation.

Suitable dosage ranges for oral administration are dependent on the potency of the particular gabapentin or pregabalin drug (once cleaved from the promoiety), but can range from about 0.1 mg to about 200 mg of drug per kilogram body weight, for example, from about 1 to about 100 mg/kg-body wt. per day. When the prodrug is a prodrug of gabapentin, typical daily doses of the prodrug in adult male patients are such as to administer about 300 to about 3600 mg equivalents of gabapentin per day to the patient. When the prodrug is a prodrug of pregabalin, typical doses of the prodrug are such as to administer about 100 to about 1200 mg equivalents of pregabalin per day to the patient. Dosage ranges may be readily determined by methods known to the skilled artisan.

### 5. EXAMPLES

The compounds, pharmaceutical compositions, and methods of treatment of the present disclosure are further defined by reference to the following examples, which describe in detail, preparation of sustained release dosage form and methods for using gabapentin and pregabalin prodrugs to treat premature ejaculation. It will be apparent to those skilled in the art that many modifications, both to materials and methods, may be practiced without departing from the scope of the invention.

### 5.1 Example 1

### Preparation of a Sustained Release Oral Dosage Form of 1-{[(α-Isobutanoyloxyethoxy)carbonyl]aminomethyl}-1-Cyclohexane Acetic Acid (100)

Oral sustained release dosage form tablets containing compound (100) were made having the ingredients shown in Table I:

TABLE I

| | | Amount/ | % | |
|---|---|---|---|---|
| | | Tablet | Composition | Ingredient |
| Ingredient | Manufacturer | (mg/tablet) | (w/w) | category |
| Compound | XenoPort (Santa | 600.00 | 45.80 | Prodrug |
| (100) | Clara, CA) | | | |
| Dibasic | Rhodia (Chicago, | 518.26 | 39.56 | Diluent |
| Calcium | IL) | | | |
| Phosphate | | | | |
| Glyceryl | Gattefosse (Saint | 60.05 | 4.58 | Lubricant/ |
| Behenate, | Pirest, Cedex, | | | Release |
| NF | France) | | | controlling |
| | | | | agent |
| Talc, USP | Barrett Minerals | 80.02 | 6.11 | Anti- |
| | (Mount Vernon, | | | adherent |
| | IN) | | | |
| Colloidal | Cabot (Tuscola, | 5.43 | 0.41 | Glidant |
| Silicon | IL) | | | |
| Dioxide, NF | | | | |
| Sodium | Fisher (Fairlawn, | 24.00 | 1.84 | Surfactant |
| Lauryl | NJ) | | | |
| Sulfate, NF | | | | |
| Magnesium | Mallinckrodt | 22.22 | 1.69 | Lubricant |
| Stearate, NF | (Phillipsburg, NJ) | | | |
| Total Weight | | 1310.00 | 100.00 | |

The tablets were made according to the following steps. Compound (100), dibasic calcium phosphate, glyceryl behenate, talc, and colloidal silicon dioxide were weighed out, screened through a #20 mesh screen and mixed in a V-blender for 15 minutes. The slugging portion of the sodium lauryl sulfate was weighed and passed through a #30 mesh screen. The slugging portion of the magnesium stearate was weighed and passed through a #40 mesh screen. Screened sodium lauryl sulfate and magnesium stearate were added to the V-blender and blended for 5 minutes. The blend was discharged and compressed into slugs of approximately 400 mg weight on a tablet compression machine. The slugs were then passed through a Comil 194 Ultra mill (Quadro Engineering, Inc., Millburn, N.J.) to obtain the milled material for further compression. The tableting portion of the sodium lauryl sulfate was weighed and passed through a #30 mesh screen. The tableting portion of the magnesium stearate was weighed and passed through a #40 mesh screen. The milled material and the tableting portions of the sodium lauryl sulfate and magnesium stearate were added to the V-blender and blended for 3 minutes. The blended material was discharged and compressed to form tablets having a total weight of 1310 mg and a compound (100) loading of 600 mg (45.8 wt %). The tablets had a mean final hardness of 16.1 to 22.2 kp (158 to 218 Newtons).

### 5.2 Example 2

### Preparation of a Sustained Release Oral Dosage Form of 3-{[(α-Isobutanoyloxyethoxy)carbonyl]aminomethyl}-5-methyl Hexanoic Acid (101)

Oral sustained release dosage form tablets containing compound (101) are made having the ingredients shown in Table II:

TABLE II

| | | Amount/ | % | |
|---|---|---|---|---|
| | | Tablet | Composition | Ingredient |
| Ingredient | Manufacturer | (mg/tablet | (w/w) | category |
| Compound | XenoPort (Santa | 600.00 | 45.80 | Prodrug |
| (101) | Clara, CA) | | | |
| Dibasic | Rhodia (Chicago, | 518.26 | 39.56 | Diluent |
| Calcium | IL) | | | |
| Phosphate | | | | |
| Glyceryl | Gattefosse (Saint | 60.05 | 4.58 | Lubricant/ |
| Behenate, NF | Pirest, Cedex, | | | Release |
| | France) | | | controlling |
| Talc, USP | Barrett Minerals | 80.02 | 6.11 | Anti- |
| | (Mount Vernon, | | | adherent |
| | IN) | | | |
| Colloidal | Cabot (Tuscola, | 5.43 | 0.41 | Glidant |
| Silicon | IL) | | | |
| Dioxide, NF | | | | |
| Sodium Lauryl | Fisher (Fairlawn, | 24.00 | 1.84 | Surfactant |
| Sulfate, NF | NJ) | | | |
| Magnesium | Mallinckrodt | 22.22 | 1.69 | Lubricant |
| Stearate, NF | (Phillipsburg, NJ) | | | |
| Total Weight | | 1310.00 | 100.00 | |

The tablets are made according to the following steps. Compound (101), dibasic calcium phosphate, glyceryl behenate, talc, and colloidal silicon dioxide are weighed out, screened through a #20 mesh screen and mixed in a V-blender for 15 minutes. The slugging portion of the sodium lauryl sulfate is weighed and passed through a #30 mesh screen. The slugging portion of the magnesium stearate is weighed and passed through a #40 mesh screen. Screened sodium lauryl sulfate and magnesium stearate are added to the V-blender and blended for 5 minutes. The blend is discharged and compressed into slugs of approximately 400 mg weight on a tablet compression machine. The slugs are then passed through a Comil 194 Ultra mill (Quadro Engineering, Inc., Millburn, N.J.) to obtain the milled material for further compression. The tableting portion of the sodium lauryl sulfate is weighed and passed through a #30 mesh screen. The tableting portion of the magnesium stearate is weighed and passed through a #40 mesh screen. The milled material and the tableting portions of the sodium lauryl sulfate and magnesium stearate are added to the V-blender and blended for 3 minutes. The blended material is discharged and compressed to form tablets having a total weight of 1310 mg and a compound (101) loading of 600 mg (45.8 wt %). The tablets have a mean final hardness ranging from 16.1 to 22.2 kp (158 to 218 Newtons).

### 5.3 Example 3

### Administration of 1-{[(α-Isobutanoyloxyethoxy)carbonyl]-aminomethyl}-1-cyclohexane Acetic Acid (100) and 3-{[(α-Isobutanoyloxyethoxy)carbonyl]aminomethyl}-5-methyl Hexanoic Acid (101) for the Treatment of Premature Ejaculation in Rats

For all the sexual behavior tests, the rapid ejaculating Sprague Dawley rats, weighing 350-450 g, are used as an animal model of premature ejaculation (classified as ejaculatory latency <300 s during baseline assessment). Prior to the experiments the animals are housed in groups (2 rats per cage) under controlled 12 h light-dark cycle (lights on at 07:00), constant temperature (23±1° C.), and humidity (55±5%). The animals are given free access to standard food pellets and water.

The rats are placed in an observation arena (50-60 cm diameter), starting 5 hours into the dark cycle and observed under red illumination. Three to four minutes after placing the male in the arena, a receptive female (ovariectomised, oestradiol benzoate/progesterone injection 48 hour before behavioral study) is introduced into the arena and the following parameters are noted: (i) ejaculatory latency (EJL; time taken from addition of receptive female into the arena to ejaculation); (ii) copulatory efficiency (CE; ejaculatory latency/the number of intromissions to ejaculation, i.e., the number of seconds between intromissions); (iii) intromission frequency (IF; the number of intromissions to ejaculation); (iv) mount frequency (MF; the number of mounts to ejaculation); (v) post ejaculatory interval (PEI; the time taken from ejaculation to the commencement of copulatory behavior).

### Delayed Ejaculation in the Presence of Compound (100) or (101)

Compounds (100) and (101) each significantly delays p-chloroamphetamine (PCA)-induced ejaculation in anaesthetized rats. The compounds are dosed orally by taking the tablets of Examples 1 and 2, finely crushing them and suspending the resulting powder in an aqueous vehicle for oral administration to rats by gavage. Compounds (100) and (101) are tested at 1, 3, 10 or 30 mg/kg po, 60 min prior to PCA administration. Compounds (100) and (101) each dose-dependently increases ejaculatory latency by up to 250%. As a baseline comparison, some of the rats are given a placebo formulation similar to Examples 1 and 2, but containing no prodrug. The excipient-only treated animals ejaculate in about 300 s, whereas animals treated with compound (100) or compound (101) at doses above 3 mg/kg po, display significantly longer ejaculatory latency to about 1000 s. The quality of erection is not influenced by either compound (100) or (101).

Using a rodent model of ejaculatory, that reflects human ejaculatory physiology, we show that compounds (100) and (101) delay ejaculation. Moreover, the study shows that gabapentin and pregabalin prodrugs will be useful in the treatment of premature ejaculation by delaying ejaculation.

### Effect of Compounds (100) and (101) on Copulatory Behavior in Rapid Ejaculating Rats

Rodent copulatory behavior is characterized by a series of mounts, with and without vaginal insertion (50-80% of mounts result in intromission (vaginal penetration)) and ejaculation occurs after 6 to 12 intromissions. Each intromission lasts a matter of seconds—it is not possible to quantify intromission length i.e., intravaginal latency. The effect of compounds (100) and (101) is assessed on a number of copulatory parameters (see above). We are focusing on ejaculatory latency as an objective clinical outcome measure of time taken to achieve ejaculation. The study is performed in rapid ejaculating rats as a model of premature ejaculation (rats characterized by ejaculatory latency <300 s at baseline). Compound (100) and (101) increases ejaculatory latency by 58% in rapidly-ejaculating conscious rats (P<0.01), i.e., compound (100) and (101) (each to be dosed at 10 mg/kg, 60 min post oral dosing)-treated animals take about 200 s to ejaculate compared to about 140 s in excipient-only treated animals. There are no other significant effects on copulatory behavior.

Using a conscious rapid-ejaculating rodent model of premature ejaculation that reflects human premature ejaculation pathophysiology, we show that compounds (100) and (101) are useful in the treatment of premature ejaculation by delaying ejaculation.

It will be apparent to those skilled in the art that many modifications, both to materials and methods, may be practiced without departing from the scope of this disclosure. Accordingly, the present embodiments are to be considered as illustrative and not restrictive, and the invention is not to be limited to the details given herein, but may be modified within the scope and equivalents of the appended claims.

What is claimed is:

1. A method of treating premature ejaculation in a male patient in need of such treatment, comprising administering to the patient a therapeutically effective amount of at least one of gabapentin prodrug of Formula (I), a pregabalin prodrug of Formula (II): 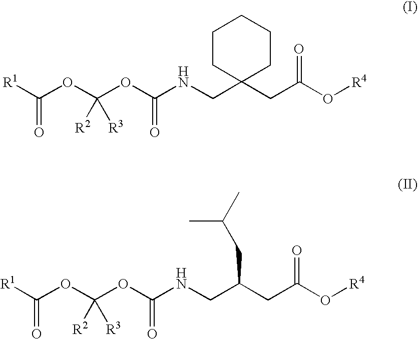
a pharmaceutically acceptable salt of any of the foregoing, a pharmaceutically acceptable solvate of any of the foregoing, and a pharmaceutically acceptable N-oxide of any of the foregoing,
wherein:
R¹ is chosen from acyl, substituted acyl, alkyl, substituted alkyl, aryl, substituted aryl, arylalkyl, substituted arylalkyl, cycloalkyl, substituted cycloalkyl, cycloheteroalkyl, substituted cycloheteroalkyl, heteroalkyl, substituted heteroalkyl, heteroaryl, substituted heteroaryl, heteroarylalkyl, and substituted heteroarylalkyl;
R² and R³ are each independently chosen from hydrogen, alkyl, substituted alkyl, alkoxycarbonyl, substituted alkoxycarbonyl, aryl, substituted aryl, arylalkyl, substituted arylalkyl, carbamoyl, substituted carbamoyl, cycloalkyl, substituted cycloalkyl, heteroaryl, substituted heteroaryl, heteroarylalkyl, and substituted heteroarylalkyl, or R² and R³ together with the carbon atom to which they are bonded form a cycloalkyl, substituted cycloalkyl, cycloheteroalkyl, or substituted cycloheteroalkyl ring; and
R⁴ is chosen from hydrogen, alkyl, substituted alkyl, aryl, substituted aryl, arylalkyl, substituted arylalkyl, cycloalkyl, substituted cycloalkyl, cycloheteroalkyl, substituted cycloheteroalkyl, heteroalkyl, substituted heteroalkyl, heteroaryl, substituted heteroaryl, heteroarylalkyl, and substituted heteroarylalkyl.

2. The method of claim 1, wherein the prodrug is administered in an amount ranging from about 10 mg to about 5000 mg per day.

3. The method of claim 1, wherein the prodrug is administered on an as needed basis.

4. The method of claim 1, wherein the prodrug is administered about 0.25 to about 20 hours before anticipated sexual activity.

5. The method of claim 1, wherein the prodrug is administered orally, parenterally, subcutaneously, intravenously, intramuscularly, transdermally, intraperitoneally, intranasally, instillationally, intracavitarally, intravesical instillationally, intraocularly, intraarterially, intralesionally, or by application to mucous membranes.

6. The method of claim 1, wherein the prodrug is a gabapentin prodrug chosen from 1-{[(α-acetoxyethoxy)carbonyl]aminomethyl}-1-cyclohexane acetic acid, 1-{[(α-propanoyloxyethoxy)carbonyl]aminomethyl}-1-cyclohexane acetic acid, 1-{[(α-butanoyloxyethoxy)carbonyl]aminomethyl}-1-cyclohexane acetic acid, 1-{[(α-isobutanoyloxyethoxy)carbonyl]aminomethyl}-1-cyclohexane acetic acid, 1-{[(α-pivaloxyethoxy)carbonyl]aminomethyl}-1-cyclohexane acetic acid, 1-{[(α-acetoxymethoxy)carbonyl]aminomethyl}-1-cyclohexane acetic acid, 1-{[(α-propanoyloxymethoxy)carbonyl]aminomethyl}-1-cyclohexane acetic acid, 1-{[(α-butanoyloxymethoxy)carbonyl]aminomethyl}-1-cyclohexane acetic acid, 1-{[(α-isobutanoyloxymethoxy)carbonyl]aminomethyl}-1-cyclohexane acetic acid, 1-{[(α-pivaloxymethoxy)carbonyl]aminomethyl}-1-cyclohexane acetic acid, 1-{[(α-acetoxypropoxy)carbonyl]aminomethyl}-1-cyclohexane acetic acid, 1-{[(α-propanoyloxypropoxy)carbonyl]aminomethyl}-1-cyclohexane acetic acid, 1-{[(α-butanoyloxypropoxy)carbonyl]aminomethyl}-1-cyclohexane acetic acid, 1-{[(α-isobutanoyloxypropoxy)carbonyl]aminomethyl}-1-cyclohexane acetic acid, 1-{[(α-pivaloxypropoxy)carbonyl]aminomethyl}-1-cyclohexane acetic acid, 1-{[(α-acetoxyisopropoxy)carbonyl]aminomethyl}-1-cyclohexane acetic acid, 1-{[(α-propanoyloxyisopropoxy)carbonyl]aminomethyl}-1-cyclohexane acetic acid, 1-{[(α-butanoyloxyisopropoxy)carbonyl]aminomethyl}-1-cyclohexane acetic acid, 1-{[(α-isobutanoyloxyisopropoxy)carbonyl]aminomethyl}-1-cyclohexane acetic acid, 1-{[(α-pivaloxyisopropoxy)carbonyl]aminomethyl}-1-cyclohexane acetic acid, 1-{[(α-acetoxybutoxy)carbonyl]aminomethyl}-1-cyclohexane acetic acid, 1-{[(α-propanoyloxybutoxy)carbonyl]aminomethyl}-1-cyclohexane acetic acid, 1-{[(α-butanoyloxybutoxy)carbonyl]aminomethyl}-1-cyclohexane acetic acid, 1-{[(α-isobutanoyloxybutoxy)carbonyl]aminomethyl}-1-cyclohexane acetic acid and 1-{[(α-pivaloxybutoxy)carbonyl]aminomethyl}-1-cyclohexane acetic acid, or a pharmaceutically acceptable salt thereof, a pharmaceutically acceptable solvate of any of the foregoing, a pharmaceutically acceptable N-oxide of any of the foregoing.

7. The method of claim 6, wherein the gabapentin prodrug is chosen from 1-{[(α-isobutanoyloxyethoxy)carbonyl]aminomethyl}-1-cyclohexane acetic acid, a pharmaceutically acceptable salt thereof, a pharmaceutically acceptable solvate of any of the foregoing, and a pharmaceutically acceptable N-oxide of any of the foregoing.

8. The method of claim 1, wherein the prodrug is a pregabalin prodrug chosen from 3-{[(α-acetoxyethoxy)carbonyl]aminomethyl}-5-methyl hexanoic acid, 3-{[(α-propanoyloxyethoxy)carbonyl]aminomethyl}-5-methyl hexanoic acid, 3-{[(α-butanoyloxyethoxy)carbonyl]aminomethyl}-5-methyl hexanoic acid, 3-{[(α-isobutanoyloxyethoxy)carbonyl]aminomethyl}-5-methyl hexanoic acid, 3-{[(α-pivaloxyethoxy)carbonyl]aminomethyl}-5-methyl hexanoic acid, 3-{[(α-acetoxymethoxy)carbonyl]aminomethyl}-5-methyl hexanoic acid, 3-{[(α-propanoyloxymethoxy)carbonyl]aminomethyl}-5-methyl hexanoic acid, 3-{[(α-butanoyloxymethoxy)carbonyl]aminomethyl}-5-methyl hexanoic acid, 3-{[(α-isobutanoyloxymethoxy)carbonyl]aminomethyl}-5-methyl hexanoic acid, 3-{[(α-pivaloxymethoxy)carbonyl]aminomethyl}-5-methyl hexanoic acid, 3-{[(α-acetoxypropoxy)carbonyl]aminomethyl}-5-methyl hexanoic acid, 3-{[(α-propanoyloxypropoxy)carbonyl]aminomethyl}-5-methyl hexanoic acid, 3-{[(α-butanoyloxypropoxy)carbonyl]aminomethyl}-5-methyl hexanoic acid, 3-{[(α-isobutanoyloxypropoxy)carbonyl]aminomethyl}-5-methyl hexanoic acid, 3-{[(α-pivaloxypropoxy)carbonyl]aminomethyl}-5-methyl hexanoic acid, 3-{[(α-acetoxyisopropoxy)carbonyl]aminomethyl}-5-methyl hexanoic acid, 3-{[(α-propanoyloxyisopropoxy)carbonyl]aminomethyl}-5-methyl hexanoic acid, 3-{[(α-butanoyloxyisopropoxy)carbonyl]aminomethyl}-5-methyl hexanoic acid, 3-{[(α-isobutanoyloxyisopropoxy)carbonyl]aminomethyl}-5-methyl hexanoic acid, 3-{[(α-pivaloxyisopropoxy)carbonyl]aminomethyl}-5-methyl hexanoic acid, 3-{[(α-acetoxybutoxy)carbonyl]aminomethyl}-5-methyl hexanoic acid, 3-{[(α-propanoyloxybutoxy)carbonyl]aminomethyl}-5-methyl hexanoic acid, 3-{[(α-butanoyloxybutoxy)carbonyl]aminomethyl}-5-methyl hexanoic acid, 3-{[(α-isobutanoyloxybutoxy)carbonyl]aminomethyl}-5-methyl hexanoic acid and 3-{[(α-pivaloxybutoxy)carbonyl]aminomethyl}-5-methyl hexanoic acid 3-{[(α-acetoxyethoxy)carbonyl]aminomethyl}-5-methyl hexanoic acid, 3-{[(α-propanoyloxyethoxy)carbonyl]aminomethyl}-5-methyl hexanoic acid, 3-{[(α-butanoyloxyethoxy)carbonyl]aminomethyl}-5-methyl hexanoic acid, 3-{[(α-isobutanoyloxyethoxy)carbonyl]aminomethyl}-5-methyl hexanoic acid, 3-{[(α-pivaloxyethoxy)carbonyl]aminomethyl}-5-methyl hexanoic acid, 3-{[(α-acetoxymethoxy)carbonyl]aminomethyl}-5-methyl hexanoic acid, 3-{[(α-propanoyloxymethoxy)carbonyl]aminomethyl}-5-methyl hexanoic acid, 3-{[(α-butanoyloxymethoxy)carbonyl]aminomethyl}-5-methyl hexanoic acid, 3-{[(α-isobutanoyloxymethoxy)carbonyl]aminomethyl}-5-methyl hexanoic acid, 3-{[(α-pivaloxymethoxy)carbonyl]aminomethyl}-5-methyl hexanoic acid, 3-{[(α-acetoxypropoxy)carbonyl]aminomethyl}-5-methyl hexanoic acid, 3-{[(α-propanoyloxypropoxy)carbonyl]aminomethyl}-5-methyl hexanoic acid, 3-{[(α-butanoyloxypropoxy)carbonyl]aminomethyl}-5-methyl hexanoic acid, 3-{[(α-isobutanoyloxypropoxy)carbonyl]aminomethyl}-5-methyl hexanoic acid, 3-{[(α-pivaloxypropoxy)carbonyl]aminomethyl}-5-methyl hexanoic acid, 3-{[(α-acetoxyisopropoxy)carbonyl]aminomethyl}-5-methyl hexanoic acid, 3-{[(α-propanoyloxyisopropoxy)carbonyl]aminomethyl}-5-methyl hexanoic acid, 3-{[(α-butanoyloxyisopropoxy)carbonyl]aminomethyl}-5-methyl hexanoic acid, 3-{[(α-isobutanoyloxyisopropoxy)carbonyl]aminomethyl}-5-methyl hexanoic acid, 3-{[(α-pivaloxyisopropoxy)carbonyl]aminomethyl}-5-methyl hexanoic acid, 3-{[(α-acetoxybutoxy)carbonyl]aminomethyl}-5-methyl hexanoic acid, 3-{[(α-propanoyloxybutoxy)carbonyl]aminomethyl}-5-methyl hexanoic acid, 3-{[(α-butanoyloxybutoxy)carbonyl]aminomethyl}-5-methyl hexanoic acid, 3-{[(α-isobutanoyloxybutoxy)carbonyl]aminomethyl}-5-methyl hexanoic acid, and 3-{[(α-pivaloxybutoxy)carbonyl]aminomethyl}-5-methyl hexanoic acid, or a pharmaceutically acceptable salt thereof, a pharmaceutically acceptable solvate of any of the foregoing, or a pharmaceutically acceptable N-oxide of any of the foregoing.

9. The method of claim 8, wherein the pregabalin prodrug is chosen from 3-{[(α-isobutanoyloxyethoxy)carbonyl]aminomethyl}-5-methyl hexanoic acid, a pharmaceutically acceptable salt thereof, a pharmaceutically acceptable solvate of any of the foregoing, and a pharmaceutically acceptable N-oxide of any of the foregoing.

10. The method of claim 1, wherein the prodrug is administered orally.

11. The method of claim 1, wherein the prodrug is administered in a sustained release oral dosage form.

12. The method of claim 11, wherein the dosage form releases the prodrug over a sufficiently long period of time to achieve a therapeutic concentration of gabapentin or pregabalin in the patient's blood plasma for a period of at least about 4 hours after swallowing the dosage form.

13. The method of claim 11, wherein the dosage form is chosen from an osmotic dosage form, a prodrug-releasing polymer, a prodrug-releasing lipid matrix, a prodrug-releasing wax, a timed-release pill, and a prodrug releasing bead.

14. The method of claim 1, wherein the prodrug is formulated with a pharmaceutically acceptable vehicle.
